(19) United States
(12) Patent Application Publication
(10) Pub. No.: US 2021/0394166 A1
(43) Pub. Date: Dec. 23, 2021

(54) CATALYST COMPOSITION

(71) Applicant: UNIVERSITY COLLEGE CARDIFF CONSULTANTS LTD, Cardiff South Wales (GB)

(72) Inventors: Benjamin David WARD, Cardiff South Glamorgan (GB); Darren Michael Charles OULD, St Austell Cornwall (GB); Mohammed Ahmed Abed BAHILI, Basrah (IQ)

(21) Appl. No.: 17/282,528

(22) PCT Filed: Oct. 3, 2019
(86) PCT No.: PCT/GB2019/052794

§ 371 Date: Apr. 2, 2021

(30) Foreign Application Priority Data
Oct. 4, 2018 (GB) 181678.6

(51) Int. Cl. B01J 31/22; C08G 63/08; C08G 63/84; C08G 63/82; C08G 59/02; C08G 59/12; C07F 5/06; C07F 7/28; C07F 3/06; C07F 11/00; C07F 15/06

(52) U.S. Cl. CPC B01J 31/2217; B01J 2531/26; C08G 63/84; C08G 63/823; C08G 59/022; C08G 59/12; C07F 5/069; C07F 7/28; C07F 3/06; C07F 11/005; C07F 15/065; B01J 2231/20; B01J 2531/31; B01J 2531/46; B01J 2531/62; B01J 2531/845; C08G 63/08

(57) ABSTRACT

Compounds can be used as catalysts, particularly in ring-opening polymerization reactions, including ring-opening co-polymerization (ROCOP) reactions, or in isocyanate trimerization reactions. The compounds have the formula L-M-Xₙ, where L is a pyridyl-bis(iminophenolate) ligand, M is a metal ion, X is a co-ligand to balance the charge of the compound, and n is an integer from 0 to 7. The compounds can be prepared by base condensation of a pyridyl-diamine compound with an aldehyde or ketone.

### TECHNICAL FIELD

The present invention relates generally to compounds that can be used as catalysts, particularly compounds that can be used as catalysts in ring-opening polymerization reactions, ring-opening co-polymerization (ROCOP) reactions and/or isocyanate trimerization reactions. The present invention also relates to the use of these compounds as catalysts, particularly in ring-opening polymerization reactions, ring-opening co-polymerization (ROCOP) reactions and/or isocyanate trimerization reactions and methods for making these compounds. The present invention additionally relates to the pyridyl-bis(iminophenolate) ligands used in the compounds disclosed herein.

### BACKGROUND

Isocyanurates are widely used as cross-linking agents in polyurethanes, affording rigid foams that are ubiquitously used in building insulation panels. Isocyanurates can be prepared by trimerizing isocyanates using nucleophilic reagents such as amines.

However, these processes employ high temperatures and require high catalyst loadings, often as much as 5-10 mol %. Under such conditions, undesirable side reactions can be prolific, reducing the overall efficiency of the production process.

Ring-opening polymerization reactions, for example using cyclic anhydrides, epoxides or cyclic esters, are a form of chain-growth polymerization where the terminal end of a polymer chain acts as a reactive centre for further monomers to react. These reactions can be used with many different substrates to make many different polymers and are very versatile.

It is therefore desirable to provide alternative and/or improved catalysts suitable for use in isocyanate trimerization and ring-opening polymerization reactions.

### SUMMARY

In accordance with a first aspect of the present invention there is provided a compound having the formula:

L-M-Xₙ

wherein:
M is a metal atom;
L is a ligand according to formula (I)

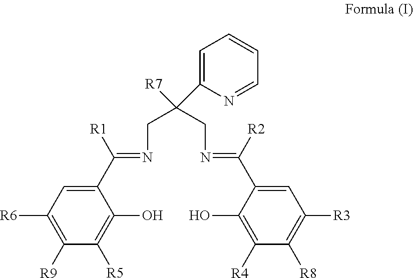

wherein,
R1, R2, R3, R4, R5, R6, R7, R8 and R9 are functional groups;
X is a co-ligand to balance the charge of the compound; and
n is an integer from 0 to 7.

Alternatively, in accordance with a further aspect of the present invention there is provided a compound having the formula:

L-M-Xₙ

wherein:
M is a metal atom;
L is a ligand according to formula (I)

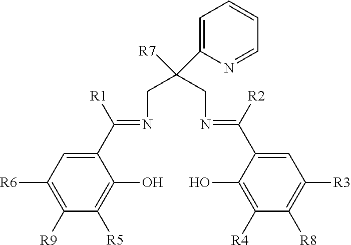

wherein,
R1 and R2 are the same or different and are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3, R4, R5, R6, R8 and R9 are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R7 is hydrogen, optionally substituted C1-C8 linear straight chain or branched alkyl such as (CH₂)_{y}OR′ and (CH₂)_{y}NR′₂, CN (nitrile) and R′₃N— (amine), where each R′ is independently hydrogen, hydroxyl, halogen, C1-C8 alkoxyl, C1-C8 linear straight chain or branched alkyl, phenyl, C2-C8 alkenyl or C2-C8 alkynyl, and y is an integer from 1 to 8;
X is a co-ligand to balance the charge of the compound; and
n is an integer from 0 to 7.

In accordance with a second aspect of the present invention there is provided the use of a compound according to any aspect or embodiment of the present invention as a catalyst in a chemical reaction. In certain embodiments, the reaction is an isocyanate trimerization. In certain embodiments, the reaction is a ring-opening polymerization.

In accordance with a third aspect of the present invention there is provided a method of making a compound according to any aspect or embodiment of the present invention.

In accordance with a fourth aspect of the present invention there is provided a ligand according to formula (I)

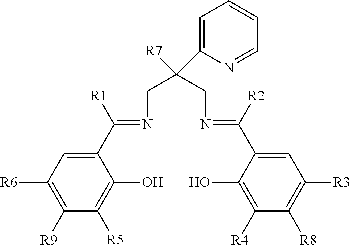

wherein R1, R2, R3, R4, R5, R6, R7, R8 and R9 are functional groups.

Alternatively, in accordance with a further aspect of the present invention there is provided a ligand according to formula (I)

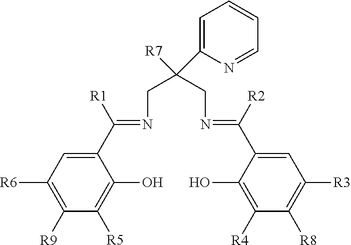

wherein,
R1 and R2 are the same or different and are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3, R4, R5, R6, R8 and R9 are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl; and
R7 is hydrogen, optionally substituted C1-C8 linear straight chain or branched alkyl such as (CH₂)_{y}OR′ and (CH₂)_{y}NR′₂, CN (nitrile) and R′₃N— (amine), where each R′ is independently hydrogen, hydroxyl, halogen, C1-C8 alkoxyl, C1-C8 linear straight chain or branched alkyl, phenyl, C2-C8 alkenyl or C2-C8 alkynyl, and y is an integer from 1 to 8.

In accordance with a fifth aspect of the present invention there is provided the use of a ligand of formula (I) according to any aspect or embodiment of the present invention in a catalyst composition.

In accordance with a sixth aspect of the present invention there is provided a method of making a ligand according to formula (I).

In accordance with a seventh aspect of the present invention there is provided the intermediate products of the method of making a compound or ligand according to any aspect or embodiment of the present invention.

In certain embodiments of any aspect or embodiment of the present invention, the following compound may be excluded:

compound according L-M-Xₙ as described herein wherein M is Cu, X is MeOH, n is 1, R1, R2, R3, R4, R5, R6, R8 and R9 are H, and R7 is methyl.

In certain embodiments of any aspect or embodiment of the present invention, M is not copper and/or M is not iron.

Certain embodiments of any aspect of the present invention may provide one or more of the following advantages:
good or improved efficiency of catalysis;
good or improved activity for catalysis;
good or improved % conversion of substrates;
good or improved catalyst loading;
good or improved reaction selectivity;
improved energy consumption (e.g. by reduced temperature);
good or improved time to complete catalysis;
good variety of available substrates;
use of Earth abundant metals having low toxicity, high terrestrial abundance meaning that they are less expensive, and the requirement to reprocess the metal via complex recycling processes is less critical:
good or improved environmentally-friendly nature of catalyst.

The details, examples and preferences provided in relation to any particulate one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION OF THE FIGURES

The invention may be described with reference to the following non-limiting FIGURES in which:

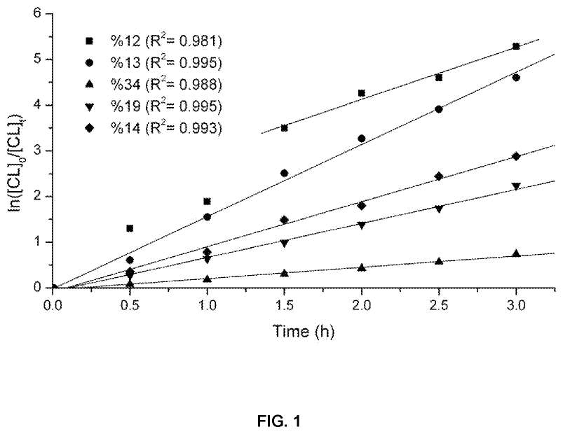

FIG. 1 shows the rate of polymerization for compounds 12, 13, 14, 19 and 34 in the ring-opening polymerization of ε-caprolactone reaction described in Example 5.

### DETAILED DESCRIPTION

Compound and Ligands

There is provided herein a compound having the formula L-M-Xₙ, where L is a pyridyl-bis(iminophenolate) ligand, M is a metal atom, X is a co-ligand to balance the charge of the compound, and n is the number of X co-ligands. There is further provided herein a pyridyl-bis(iminophenolate) ligand, L.

The compounds may be referred to as coordination complexes or metal complexes, which comprise or consist of a central metal atom surrounded by chemically bound ligands. The ligand, L, may be a polydentate ligand (a ligand that bonds to the central metal atom through more than one of the ligand's atoms). The ligand, L, may, for example, bind to the central metal atom through 1, 2, 3, 4, 5 or 6 of the ligand's atoms. Preferably, the ligand may be able to bind to the central metal atom through at least 5 of the ligand's atoms, particularly the 3 nitrogen atoms (2 imine N atoms and 1 pyridinyl N atom) and/or the 2 oxygen atoms (O atoms of the OH groups of the phenol rings). The complexes may, for example, be 5 or 6-coordinate. The ligand may therefore have an octahedral, trigonal bipyramidal or square pyramidal geometry, for example depending on how many X co-ligands are present. The ligand, L, may be hemi-labile, meaning that one or more of the bonds between the central metal atom and the ligand, L, may be easily broken whilst one or more of the other bonds remain.

It has surprisingly been found that the compounds provided herein, which include ligand L, are useful catalysts, particularly in isocyanate trimerization and ring-opening polymerization reactions.

The term “optionally substituted” may refer to a substitution of a hydrogen atom with any functional group. The substituted functional group may, for example, be hydroxy, C1-C8 linear or branched chain alkyl group, C1-C8 linear or branched chain alkenyl group, C1-C8 linear or branched chain alkynyl group, C1-C8 alkoxy, ═NH, —NH₂, R′₃N— (amine) where R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl or C3-C12 aryl group such as phenyl. Where the optional substitution occurs on a ring, the optional substitution may occur at the para position.

The term “halogen” may refer to any of F, Cl, Br, I and At. In certain embodiments, the halogen is Cl or Br.

The term “C1-C8 alkoxy” refers to any group having the structure —OR, where R is a linear or branched alkyl having 1 to 8 carbon atoms. In certain embodiments, the C1-C8 alkoxy may be a C1-C4 alkoxy.

In certain embodiments, the “C1-C8 linear straight chain or branched alkyl” may be a C1-C4 linear straight chain or branched alkyl.

In certain embodiments, the “C3-C15 cycloalkyl” may be a C5-C12 cycloalkyl, for example a C5-C10 cycloalkyl.

In certain embodiments, the “C2-C8” alkenyl is a C2-C4 alkenyl. In certain embodiments, the “C2-C8 alkynyl” is a C2-C4 alkynyl.

In certain embodiments the “phenyl-V—(C1-C8)-alkyl where V is selected from a single bond, O, S and NH” is phenyl-V—(C1-C4)-alkyl where V is selected from a single bond, O, S and NH. In certain embodiments, the “phenyl-V—(C1-C8)-alkyl where V is selected from a single bond, O, S and NH” is phenyl-V—(C1-C8)-alkyl is phenyl-V—(C1-C8)-alkyl where V is selected from a single bond and O. In certain embodiments, the “phenyl-V—(C1-C8)-alkyl where V is selected from a single bond, O, S and NH” is phenyl-V—(C1-C8)-alkyl is phenyl-V—(C1-C4)-alkyl where V is selected from a single bond and O.

In certain embodiments, the “phenyl-(C1-C8)-alkyl-V— where V is selected from a single bond, O, S and NH” is phenyl-(C1-C4)-alkyl-V— where V is selected from a single bond, O, S and NH. In certain embodiments, the “phenyl-(C1-C8)-alkyl-V— where V is selected from a single bond, O, S and NH” is phenyl-(C1-C8)-alkyl-V— where V is selected from a single bond and O. In certain embodiments, the “phenyl-(C1-C8)-alkyl-V— where V is selected from a single bond, O, S and NH” is phenyl-(C1-C4)-alkyl-V— where V is selected from a single bond and O.

M

M is a metal atom. The metal atom may, for example, be a metal cation.

The metal atom may, for example, have any oxidation state suitable to make a metal complex as described herein, for example suitable for use as a catalyst, for example a catalyst for isocyanate trimerization and/or ring-opening polymerization. For example, the metal atom may have an oxidation state of (II) or (Ill) or (IV). In other words, the metal may have a charge of +2, +3 or +4.

The metal atom may be any metal suitable to make a metal complex as described herein, for example suitable for use as a catalyst, for example a catalyst for isocyanate trimerization and/or ring-opening polymerization.

The metal may, for example, be selected from scandium, titanium, chromium, manganese, iron, cobalt, nickel, copper, zinc, aluminium, gallium, yttrium, zirconium, niobium, molybdenum, technetium, ruthenium, rhodium, palladium, silver, cadmium, hafnium, tantalum, tungsten, rhenium, osmium, iridium, platinum, gold, thallium and lead. For example, the metal is selected from titanium, chromium, iron, cobalt, copper, aluminium and gallium. For example, the metal is selected from titanium, chromium, cobalt, aluminium and gallium. For example, the metal may be aluminium.

The metal M may, for example, be any metal atom excluding copper.

For example, when X is methanol, n is 1, R1 to R6, R8 and R9 are H and R7 is methyl, M is any metal atom excluding copper.

L

L is a ligand according to formula (I):

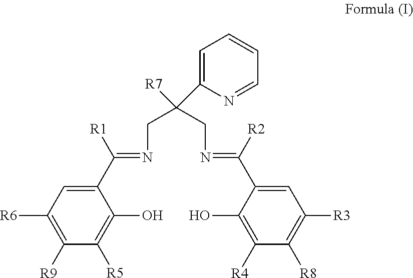

wherein,
R1 to R9 are functional groups. R1 to R9 may, for example, be any functional group suitable to make suitable to make a metal complex as described herein, for example suitable for use as a catalyst, for example a catalyst for isocyanate trimerization and/or ring-opening polymerization.

In certain embodiments:
R1 and R2 are the same or different and are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3, R4, R5, R6, R8 and R9 are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl; and
R7 is hydrogen, optionally substituted C1-C8 linear straight chain or branched alkyl such as (CH₂)_{y}OR′ and (CH₂)_{y}NR′₂, CN (nitrile) and R′₃N— (amine), where each R′ is independently hydrogen, hydroxyl, halogen, C1-C8 alkoxyl, C1-C8 linear straight chain or branched alkyl, phenyl, C2-C8 alkenyl or C2-C8 alkynyl, and y is an integer from 1 to 8.

R1 and R2

R1 and R2 may be the same or different and may each be independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl.

R1 and R2 may be the same or different and may each be independently selected from hydrogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl.

R1 and R2 may be the same or different and may each be independently selected from hydrogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl and optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl.

R1 and R2 may be the same or different and may each be independently selected from hydrogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl.

R1 and R2 may be the same or different and may each be independently selected from hydrogen and optionally substituted C1-C8 linear straight chain or branched alkyl. For example, R1 and R2 may be the same or different and may each be independently selected from hydrogen and methyl.

In all of the above embodiments, R1 and R2 may be the same. For example, R1 and R2 are both hydrogen or R1 and R2 are both optionally substituted C1-C8 linear straight chain or branched alkyl such as methyl.

Alternatively, in all of the above embodiments, R1 and R2 may be different. For example, R1 may be hydrogen and R2 may be selected from halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. For example, R1 is hydrogen and R2 is optionally substituted C1-C8 linear straight chain or branched alkyl. For example, R1 is hydrogen and R2 is methyl.

R1 and R2 may be different and may be selected from halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. For example, R1 and R2 are different and are both optionally substituted C1-C8 linear straight chain or branched alkyl.

In certain embodiments, R1 and R2 are not hydrogen. For example, wherein M is copper, X is methanol, n is 1, R3 to R6, R8 and R9 are hydrogen and R7 is methyl, R1 and R2 may not be hydrogen.

R3, R4, R5, R6, R8 and R9

R3, R4, R5, R6, R8 and R9 may each be independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may each be independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl and optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may each be independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may each be independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may be each be independently selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may be each independently selected from hydrogen, halogen, optionally substituted C1-C8 alkoxy or optionally substituted C1-C8 linear straight chain or branched alkyl and optionally substituted C3-C15 cycloalkyl. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may each be independently selected from hydrogen, halogen, optionally substituted C1-C8 alkoxy and optionally substituted C1-C8 linear straight chain or branched alkyl. For example, R3, R4, R5 and R6 are each independently selected from hydrogen, halogen, optionally substituted C1-C8 alkoxy and optionally substituted C3-C15 cycloalkyl. For example, R3, R4, R5 and R6 are each independently selected from hydrogen, halogen, optionally substituted C1-C8 linear straight chain or branched alkyl and optionally substituted C3-C15 cycloalkyl. For example, R3, R4, R5 and R6 are each independently selected from halogen, optionally substituted C1-C8 alkoxy, optionally substituted C1-C8 linear straight chain or branched alkyl and optionally substituted C3-C15 cycloalkyl. For example, R3, R4, R5 and R6 are each independently selected from hydrogen, optionally substituted C1-C8 alkoxy, optionally substituted C1-C8 linear straight chain or branched alkyl and optionally substituted C3-C15 cycloalkyl. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5, R6, R8 and R9 may each be independently selected from hydrogen, chlorine, methoxy, butyl such as tertiary-butyl, adamantyl, methoxy and optionally substituted phenyl which is optionally fused to the phenol ring in formula (I) to form a naphthalene moiety and optionally substituted C3-C15 cycloalkyl. For example, R3, R4, R5 and R6 are each independently selected from hydrogen, chlorine, methoxy, butyl such as tertiary-butyl, adamantyl and methoxy. In certain embodiments, R3 and R6 are the same and/or R4 and R5 are the same but optionally different to R3 and R6. In certain embodiments, R8 and R9 are the same but are optionally different to R3 and R6 and/or are different to R4 and R5.

R3, R4, R5 and R6 may be the same. R3, R4, R5 and R6 may be the same and may be selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl.

R3, R4, R5, R6, R8 and R9 may be the same. R3, R4, R5, R6, R8 and R9 may be the same and may be selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl.

R3, R4, R5 and R6 may be the same. R3, R4, R5 and R6 may be the same and may be selected from hydrogen, optionally substituted C1-C8 linear straight chain or branched alkyl, such as butyl, for example tertiary-butyl, and halogen, such as chlorine, fluorine, bromine or iodine, for example chlorine. For example, R3, R4, R5 and R6 are the same and are hydrogen. For example, R3, R4, R5 and R6 are the same and are optionally substituted C1-C8 linear straight chain or branched alkyl, such as butyl, such as tertiary-butyl. For example, R3, R4, R5 and R6 are the same and are halogen, such as chlorine, fluorine, bromine or iodine, for example chlorine.

R3, R4, R5 and R6 may be optionally substituted phenyl which is optionally fused to the phenol ring in formula (I) to form a naphthalene moiety.

R3, R4, R5 and R6 may be different. R3, R4, R5 and R6 may be different and may be each independently selected from hydrogen, halogen, optionally substituted C1-C8 alkoxy and optionally substituted C1-C8 linear straight chain or branched alkyl. For example, R3, R4, R5 and R6 may be different and may be each independently selected from optionally substituted C1-C8 alkoxy and optionally substituted C1-C8 linear straight chain or branched alkyl. For example, R3, R4, R5 and R6 may be different and may be each independently selected from butyl such as tertiary-butyl, methyl, adamantyl and methoxy. For example, one or both of R3 and R6 are an optionally substituted C1-C8 alkoxy such as methoxy. For example, one or both of R3 and R6 are an optionally substituted C1-C8 linear straight chain or branched alkyl such as butyl such as tertiary-butyl. For example, one or both of R3 and R6 are an optionally substituted C3-C15 cycloalkyl such as adamantyl. For example, one or both of R4 and R5 are an optionally substituted C1-C8 alkoxy such as methoxy. For example, one or both of R4 and R5 are an optionally substituted C1-C8 linear straight chain or branched alkyl such as butyl such as tertiary-butyl. For example, one or both of R4 and R5 are an optionally substituted C3-C15 cycloalkyl such as adamantyl.

R3 and R6 may be the same. R4 and R5 may be the same. R8 and R9 may be the same. R3 and R6 may be the same and R4 and R5 may be the same, but different to R3 and R6. R3 and R6 may be the same and R4 and R5 may be the same and R8 and R9 may be the same. R3 and R6 may be the same and R4 and R5 may be the same and R8 and R9 may be the same but each pair of R3 and R6, R4 and R5 and R8 and R9 may be different to one or both of the other pairs, R3 and R6, R4 and R5 and R8 and R9. R3 and R6 may be the same and selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl. Additionally or alternatively, R4 and R5 may be the same (but optionally different to R3 and R6) and selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl. Additionally or alternatively, R8 and R9 may be the same (but optionally different to R3 and R6 and/or R4 and R5) and selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl. Additionally or alternatively, R8 and R9 may be the same (but optionally different to R3 and R6 and/or R4 and R5) and selected from hydrogen and optionally substituted C1-C8 alkoxy.

R3 and R6 may be optionally substituted C1-C8 alkoxy and R4 and R5 may be optionally substituted C1-C8 linear straight chain or branched alkyl. R4 and R5 may be optionally substituted C1-C8 alkoxy and R3 and R6 may be optionally substituted C1-C8 linear straight chain or branched alkyl. R3 and R6 may be optionally substituted C1-C8 linear straight chain or branched alkyl and R4 and R5 may be optionally substituted C3-C15 cycloalkyl. R3 and R6 may be methoxy and R4 and R5 may be tertiary-butyl. R3 and R6 may be methyl and R4 and R5 may be adamantyl. For each embodiment, R8 and R9 may be hydrogen or optionally substituted C—C8 alkoxy such as methoxy.

In some embodiments, the identities of R3, R4, R5 and R6 may be selected from the following examples: (1) R3, R4, R5 and R6 are all hydrogen, (2) R3, R4, R5 and R6 are all optionally substituted C1-C8 linear straight chain or branched alkyl, such as a branched alkyl, such as tertiary-butyl, (3) R3, R4, R5 and R6 are optionally substituted phenyl which is optionally fused to the phenol ring in formula (I) to form a naphthalene moiety, (4) R3 and R6 are both optionally substituted C1-C8 alkoxy, such as methoxy, and R4 and R5 are both optionally substituted C1-C8 linear straight chain or branched alkyl, such as a branched alkyl, such as tertiary-butyl, (5) R3 and R6 are both optionally substituted C1-C8 linear straight chain or branched alkyl, such as a methyl, and R4 and R5 are optionally substituted C3-C15 cycloalkyl, such as adamantyl (6) R3, R4, R5 and R6 are all halogen, such as chlorine. For each example, R8 and R9 may be hydrogen or optionally substituted C—C8 alkoxy such as methoxy.

In certain embodiments, R3 to R6, R8 and R9 may not be hydrogen. For example, wherein M is copper, X is methanol, n is 1, R1 and R2 are hydrogen and R7 is methyl, R3 to R6, R8 and R9 may not be hydrogen.

R7

R7 may be hydrogen, optionally substituted C1-C8 linear straight chain or branched alkyl such as (CH₂)_{y}OR′ and (CH₂)_{y}NR′₂, CN (nitrile) and R′₃N— (amine), where each R′ is independently hydrogen, hydroxyl, halogen, optionally substituted C1-C8 alkoxyl, optionally substituted C1-C8 linear straight chain or branched alkyl, optionally substituted phenyl, optionally substituted C2-C8 alkenyl or optionally substituted C2-C8 alkynyl, and y is an integer from 1 to 8.

R7 may, for example, be optionally substituted C1-C8 linear straight chain alkyl. For example, the optionally substituted C1-8 linear straight chain alkyl may be C1-C8, or C1 to C7, or C1 to C6 or C1 to C5, or C1 to C4, or C1 to C3, or C1 to C2, or it may be C1. For example, the optionally substituted C1-8 linear straight chain alkyl may be C2-C8, or C2 to C7, or C2 to C6 or C2 to C5, or C2 to C4, or C2 to C3, or it may be C2. For example, the optionally substituted C1-8 linear straight chain alkyl may be C3-C8, or C3 to C7, or C3 to C6 or C3 to C5, or C3 to C4, or it may be C3. For example, the optionally substituted C1-8 linear straight chain alkyl may be C4-C8, or C4 to C7, or C4 to C6 or C4 to C5, or it may be C4. For example, the optionally substituted C1-8 linear straight chain alkyl may be C5 to C8, or C5 to C7, or C5 to C6 or it may be C5. For example, the optionally substituted C1-8 linear straight chain alkyl may be C6-C8, or C6 to C7, or it may be C6. For example, the optionally substituted C1-8 linear straight chain alkyl may be C7-C8, or it may be C7 or it may be C8.

R7 may, for example, be hydrogen or optionally substituted C1-C8 branched alkyl. R7 may, for example, be optionally substituted C1-C8 branched alkyl. For example, the optionally substituted C1-8 branched alkyl may be C1-C8, or C1 to C7, or C1 to C6 or C1 to C5, or C1 to C4, or C1 to C3, or C1 to C2, or it may be C1. For example, the optionally substituted C1-8 branched alkyl may be C2-C8, or C2 to C7, or C2 to C6 or C2 to C5, or C2 to C4, or C2 to C3, or it may be C2. For example, the optionally substituted C1-8 branched alkyl may be C3-C8, or C3 to C7, or C3 to C6 or C3 to C5, or C3 to C4, or it may be C3. For example, the optionally substituted C1-8 branched alkyl may be C4-C8, or C4 to C7, or C4 to C6 or C4 to C5, or it may be C4. For example, the optionally substituted C1-8 branched alkyl may be C5 to C8, or C5 to C7, or C5 to C6 or it may be C5. For example, the optionally substituted C1-8 branched alkyl may be C6-C8, or C6 to C7, or it may be C6. For example, the optionally substituted C1-8 branched alkyl may be C7-C8, or it may be C7 or it may be C8.

The optional substitution on the C1-C8 linear straight chain or branched alkyl may, for example, be one or more of hydroxy, OR′, ═NH, —NH₂ or —NR′₃. Thus, R7 may be (CH₂)_{y}OR′ and (CH₂)_{y}NR′₂ where each R′ is independently hydrogen, hydroxyl, halogen, C1-C8 alkoxyl, C1-C8 linear straight chain or branched alkyl, phenyl, C2-C8 alkenyl or C2-C8 alkynyl, and y is an integer from 1 to 8. For example, R7 may be (CH₂)_{y}OR′ and (CH₂)_{y}NR′₂ where each R′ is independently hydrogen, C1-C8 linear straight chain or branched alkyl or phenyl and y is an integer from 1 to 4, for example 1 or 2.

R7 may, for example, be hydrogen or C1-C8 linear straight chain or branched chain alkyl. For example, R7 is methyl. For example, R7 is propyl such as isopropyl. For example, R7 is hydrogen.

In certain embodiments R7 is selected from hydrogen and optionally substituted C1-C8 linear straight chain or branched alkyl, but excluding methyl. For example, wherein M is copper, X is methanol, n is 1, and R1 to R6 are H, R7 may be hydrogen or optionally substituted C1-C8 linear straight chain or branched alkyl, excluding methyl.

Xₙ

X is a co-ligand to balance the charge of the compound. In certain embodiments, the metal M has a charge of +2, +3 or +4 and the ligand L has a charge of −2. Therefore, in certain embodiments, X has a charge of −1 or −2 and n is 0, 1 or 2.

X may, for example, be selected from hydrogen, halogen, optionally substituted C1-C8 linear straight chain or branched alkyl, optionally substituted C1-C8 alkoxy, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, optionally substituted phenyl-V—(C1-C8-alkyl) where V is selected from a single bond, O, S and NH, optionally substituted phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R″—C(O)—, R″—C(O)—O—, R″—O—, R″2N—C(O)—O—, R″(OR″)2C—, (OR″)3C—, (OR″)3C—O—, nitro (—NO2), nitrate (—ONO2), nitrile (CN), nitrite (—ONO), nitroso (NO), cyano (—OCN or —NCO), optionally substituted pyridyl, R″(NOH)C— (oxime), R″3N— (amine), R″4N+— (quaternary amine), R″2N—C(O)— (amide), R″(NR′)—C— (imine), R′(CO)NR″—C(O)— (imide), N3 (azide), R″N2-(azo), —SH (thiol), (OH)(O)S— (sulfino), (O)(O)(OH)S— (sulfo), thiocyanate (—SCN or —NCS), (S)HC— (thial), R″S— (sulfide), R″S—S— (disulphide), R″(O)S— (sulfoxide), (O)(O)R″S— (sulfonyl), R″SC (thioketone), R″S—C(O)— (thioester), R″C(O)—S—, R″3P (phosphino), (OH)2(O)P— (phosphono), (OH)2(O)P—O— (phosphate) and (OR″)2(O)P—O— (phosphoester), where each R″ is independently hydrogen, hydroxyl, halogen, optionally substituted C1-C8 alkoxy, optionally substituted C1-C8 linear straight chain or branched alkyl, optionally substituted C2-C8 alkenyl or optionally substituted C2-C8 alkynyl.

X may, for example, be selected from hydrogen, halogen, optionally substituted C1-C8 linear straight chain or branched alkyl, optionally substituted C1-C8 alkoxy, optionally substituted C3-C15 cycloalkyl, optionally substituted phenyl, optionally substituted phenoxy, phenyl-V—(C1-C8)-alkyl where V is selected from a single bond, O, S and NH, phenyl-(C1-C8-alkyl)-V— where V is selected from a single bond, O, S and NH, and R″—O— where R″ is optionally substituted C1-C8 linear straight chain or branched alkyl.

For example, X may be selected from halogen, optionally substituted C1-C8 linear straight chain or branched alkyl, optionally substituted C1-C8 alkoxy, optionally substituted phenoxy, and phenyl-(C1-C8-alkyl)-V— where V is selected from a single bond and O. For example, X is selected from chlorine, isopropoxide, methyl, —OCH2Ph, —CH2Ph and —O-4-C6H4Me.

n is an integer from 0 to 7. For example, n may be an integer from 0 to 6, or 0 to 5, or 0 to 4, or 0 to 3, or 0 to 2, or 0 to 1, or wherein n is 0. For example, n may be an integer from 1 to 7, or 1 to 6, or 1 to 5, or 1 to 4 or 1 to 3, or 1 to 2, or wherein n is 1. For example, n may be an integer from 2 to 7, or 2 to 6, or 2 to 5, or 2 to 3, or wherein n is 2. For example, n may be an integer from 3 to 7, or 3 to 6, or 3 to 5 or 3 to 4 or wherein n is 3. For example, n may be an integer from 4 to 7, or 4 to 6 or 4 to 5 or wherein n is 4. For example, n may be an integer from 5 to 7, or 5 to 6, or wherein n is 5. For example, n may be an integer from 6 to 7, or wherein n is 6 or wherein n is 7.

In certain embodiments, n is 1 or 2. For example, n is 1.

Exemplary Combinations

Any combination of any two or more embodiments described herein for each of M, R1, R2, R3, R4, R5, R6, R7, R8, R9, X and n is envisaged for this application. In certain embodiments, these combinations exclude compounds explicitly excluded from one or more embodiments described herein. Any one or more of the embodiments described above for each of M, R1, R2, R3, R4, R5, R6, R7, R8, R9, X and n may be substituted in any of the embodiments described below.

In certain embodiments:
M is a metal atom having a charge of +3 or +4;
R1 and R2 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)2C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N—0 (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, 0, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from hydrogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6;
R4 and R5 are the same, and may be the same or different to R3 and R6, and are selected from hydrogen, halogenoptionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)2C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, 0, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is selected from hydrogen, halogen, optionally substituted C1-C8 linear straight chain or branched alkyl, optionally substituted C1-C8 alkoxy, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, optionally substituted phenyl-V—(C1-C8-alkyl) where V is selected from a single bond, O, S and NH, optionally substituted phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R″—C(O)—, R″—C(O)—O—, R″—O—, R″2N—C(O)—O—, R″(OR″)2C—, (OR″)3C—, (OR″)3C—O—, nitro (—NO2), nitrate (—ONO2), nitrile (CN), nitrite (—ONO), nitroso (NO), cyano (—OCN or —NCO), optionally substituted pyridyl, R″(NOH)C— (oxime), R″3N— (amine), R″4N+— (quaternary amine), R″2N—C(O)— (amide), R″(NR′)—C— (imine), R′(CO)NR″—C(O)— (imide), N3 (azide), R″N2- (azo), —SH (thiol), (OH)(O)S— (sulfino), (O)(O)(OH)S— (sulfo), thiocyanate (—SCN or —NCS), (S)HC— (thial), R″S— (sulfide), R″S—S— (disulphide), R″(O)S— (sulfoxide), (O)(O)R″S— (sulfonyl), R″SC (thioketone), R″S—C(O)— (thioester), R″C(O)—S—, R″3P (phosphino), (OH)2(O)P— (phosphono), (OH)2(O)P—O— (phosphate) and (OR″)2(O)P—O— (phosphoester), where each R″ is independently hydrogen, hydroxyl, halogen, optionally substituted C1-C8 alkoxy, optionally substituted C1-C8 linear straight chain or branched alkyl, optionally substituted C2-C8 alkenyl or optionally substituted C2-C8 alkynyl; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)2C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C-(oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is selected from hydrogen, halogen, optionally substituted C1-C8 linear straight chain or branched alkyl, optionally substituted C1-C8 alkoxy, optionally substituted C3-C15 cycloalkyl, optionally substituted phenyl, optionally substituted phenoxy, phenyl-V—(C1-C8)-alkyl where V is selected from a single bond, O, S and NH, phenyl-(C1-C8-alkyl)-V— where V is selected from a single bond, O, S and NH, and R″—O— where R″ is optionally substituted C1-C8 linear straight chain or branched alkyl; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from hydrogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from selected from hydrogen and optionally substituted C1-C8 linear straight chain or branched alkyl;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy and phenyl-V—(C1-C8-alkyl) where V is selected from a single bond, O, S and NH, phenyl-(C1-C8-alkyl)-V— where V is selected from a single bond, O, S and NH;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are selected from hydrogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, R′—C(O)—, R′—C(O)—O—, R′—O—, R′₂N—C(O)—O—, R′(OR′)₂C—, (OR′)₃C— and (OR′)₃C—O—, where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3 and R6 are the same and are selected from hydrogen, halogen, optionally substituted C1-C8 alkoxy or optionally substituted C1-C8 linear straight chain or branched alkyl and optionally substituted C3-C15 cycloalkyl;
R4 and R5 are the same, and may be the same or different to R3 and R6, and are selected from hydrogen, halogen, optionally substituted C1-C8 alkoxy or optionally substituted C1-C8 linear straight chain or branched alkyl and optionally substituted C3-C15 cycloalkyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are same and are selected from selected from hydrogen and optionally substituted C1-C8 linear straight chain or branched alkyl;
R3 and R6 are the same and are selected from hydrogen, halogen, optionally substituted C1-C8 alkoxy or optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R4 and R5 are the same, and may be the same or different to R3 and R6, and are selected from hydrogen, halogen, optionally substituted C1-C8 alkoxy or optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium;
R1 and R2 are the same and are same and are selected from selected from hydrogen and optionally substituted C1-C8 linear straight chain or branched alkyl;
R3 and R6 are the same and are selected from hydrogen, halogen, optionally substituted C1-C8 alkoxy or optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R4 and R5 are the same, and may be the same or different to R3 and R6, and are selected from hydrogen, halogen, optionally substituted C1-C8 alkoxy or optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is hydrogen or optionally substituted C1-C8 linear straight chain or branched alkyl;
X is selected from hydrogen, halogen, optionally substituted C1-C8 linear straight chain or branched alkyl, optionally substituted C1-C8 alkoxy, optionally substituted C3-C15 cycloalkyl, optionally substituted phenyl, optionally substituted phenoxy, phenyl-V—(C1-C8)-alkyl where V is selected from a single bond, O, S and NH, phenyl-(C1-C8-alkyl)-V— where V is selected from a single bond, O, S and NH, and R″—O— where R″ is optionally substituted C1-C8 linear straight chain or branched alkyl; and
n is 1 or 2.

In some embodiments:
M is aluminum;
X is selected from halogen such as chlorine, optionally substituted C1-C8 linear straight chain or branched alkyl such as methyl, optionally substituted C1-C8 alkoxy such as isopropoxide, optionally substituted phenoxy such as-O-4-C6H4Me, and phenyl-(C1-C8-alkyl)-V— where V is selected from a single bond and O, such as —OCH2Ph and —CH2Ph;
n is 1 or 2;
R1 and R2 are selected from hydrogen and methyl;
R3, R4, R5 and R6 are each independently selected from hydrogen, halogen such as chlorine, optionally substituted C1-C8 alkoxy such as methoxy, optionally substituted C1-C8 linear straight chain or branched alkyl such as branched alkyl such as tertiary-butyl, optionally substituted phenyl which is optionally fused to the phenol ring in formula (I) to form a naphthalene moiety, and optionally substituted C3-C15 cycloalkyl such as adamantyl;
R8 and R9 are the same, and may be the same or different to R3 and R6 and may be the same or different to R4 and R5, and are selected from hydrogen and optionally substituted C1-C8 alkoxy; and
R7 is selected from hydrogen and methyl.

In certain embodiments:
M is aluminum;
X is Cl;
n is 1;
R1 and R2 are hydrogen;
R3, R4, R5 and R6 are selected from hydrogen, methoxy, tertiary-butyl, methyl and adamantyl, such as wherein R3, R4, R5 and R6 are all hydrogen or tertiary-butyl or wherein R3 and R6 are methoxy and R4 and R5 are tertiary-butyl or wherein R3 and R6 are methyl and R4 and R5 are adamantyl;
R8 and R9 are the same, and may be the same or different to R3 and R6 and may be the same or different to R4 and R5, and are selected from hydrogen and optionally substituted C1-C8 alkoxy; and
R7 is methyl.

In certain embodiments:
M is aluminum;
X is selected from methyl, —OCH2Ph, -Me and —O-4-C6H4Me;
n is 1;
R1 and R2 are hydrogen;
R3, R4, R5 and R6 are hydrogen;
R8 and R9 are the same, and are selected from hydrogen and optionally substituted C1-C8 alkoxy; and
R7 is methyl.

In certain embodiments:
M is aluminum;
X is selected from methyl, —OCH2Ph, —CH2Ph and —OⁱPr;
n is 1;
R1 and R2 are hydrogen;
R3, R4, R5 and R6 are hydrogen, tertiary-butyl, methoxy, adamantyl, methyl and chlorine, such as wherein all of R3 to R6 are hydrogen or wherein all of R3 to R6 are tertiary-butyl or wherein all of R3 to R6 are halogen or wherein R4 and R5 are tertiary-butyl and R3 and R6 are methoxy or wherein R4 and R5 are adamantyl and R3 and R6 are methyl;
R8 and R9 are the same, and are selected from hydrogen and optionally substituted C1-C8 alkoxy; and
R7 is methyl.

In certain embodiments:
M is selected from titanium, chromium, iron, cobalt, copper, aluminium and gallium, for example, selected from titanium, chromium, cobalt, aluminium and gallium, for example M is aluminium;
R1 and R2 are hydrogen;
R3 and R6 are the same and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl. In certain embodiments, R3 and R6 are the same and/or R4 and R5 are the same but optionally different to R3 and R6;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′2N—C(O)—O—, R′(OR′)2C—, (OR′)3C—, (OR′)3C—O—, nitro (—NO2), nitrate (—ONO2), nitrile (CN), nitrite (—ONO), nitroso (NO), cyano (—OCN or —NCO), optionally substituted pyridyl, R′(NOH)C— (oxime), R′3N— (amine), R′4N+— (quaternary amine), R′2N—C(O)— (amide), R′(NR′)—C— (imine), R′(CO)NR′—C(O)— (imide), N3 (azide), R′N2- (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′3P (phosphino), (OH)2(O)P— (phosphono), (OH)2(O)P—O— (phosphate) and (OR′)2(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R8 and R9 are the same, and may be the same or different to R3 and R6 and may be the same or different to R4 and R5, and are selected from hydrogen, optionally substituted C1-C8 alkoxy and optionally substituted C1-C8 linear straight chain or branched chain alkyl;
R7 is methyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium, for example M is aluminium;
R1 and R2 are hydrogen or optionally substituted C1-C8 linear straight chain or branched alkyl
R3 and R6 are the same and are selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R8 and R9 are the same, and may be the same or different to R3 and R6 and may be the same or different to R4 and R5, and are selected from hydrogen and optionally substituted C1-C8 alkoxy;
R7 is hydrogen or optionally substituted C1-C8 linear straight chain or branched alkyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium, for example M is aluminium;
R1 and R2 are hydrogen;
R3 and R6 are the same and are selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R4 and R5 are the same, and may be the same or different to R3 and R6, and are selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl;
R8 and R9 are the same, and may be the same or different to R3 and R6 and may be the same or different to R4 and R5, and are selected from hydrogen and optionally substituted C1-C8 alkoxy;
R7 is methyl;
X is a co-ligand having a charge of −1 or −2; and
n is 1 or 2.

In certain embodiments:
M is selected from titanium, chromium, iron, cobalt, copper, aluminium and gallium, for example, selected from titanium, chromium, cobalt, aluminium and gallium, for example M is aluminium;
R1 and R2 are hydrogen;
R3 and R6 are the same and are selected from hydrogen, halogen, optionally substituted C1-C8 alkoxy, optionally substituted C1-C8 linear straight chain or branched alkyl and optionally substituted C3-C15 cycloalkyl;
R4 and R5 are the same, and may be the same or different to R3 and R6, and are selected from hydrogen, halogen, optionally substituted C1-C8 alkoxy, optionally substituted C1-C8 linear straight chain or branched alkyl and optionally substituted C3-C15 cycloalkyl;
R8 and R9 are the same, and may be the same or different to R3 and R6 and may be the same or different to R4 and R5, and are selected from hydrogen and optionally substituted C1-C8 alkoxy;
R7 is methyl;
X is a co-ligand having a charge of −1 or −2; and
n is 1 or 2.

The catalyst may, for example, be one or more of the following compounds:

| | | | | R1, | | | R8, | |
|---|---|---|---|---|---|---|---|---|
| Compound | M | X | n | R2 | R3, R6 | R4, R5 | R9 | R7 |
| 1 | Al | Cl | 1 | H | H | H | H | Methyl |
| 2 | Al | Cl | 1 | H | t-Butyl | t-Butyl | H | Methyl |
| 3 | Al | Cl | 1 | H | OMe | t-Butyl | H | Methyl |
| 4 | Al | Cl | 1 | H | Me | Adamantyl | H | Methyl |
| 5 | Al | Me | 1 | H | H | H | H | Methyl |
| 6 | Al | —OTol (—O—4—C₆H₄Me) | 1 | H | H | H | H | Methyl |
| 7 | Al | —OBn (—O—CH₂Ph) | 1 | H | H | H | H | Methyl |
| 8 | Al | Me | 1 | H | t-Butyl | t-Butyl | H | Methyl |
| 9 | Al | Me | 1 | H | OMe | t-Butyl | H | Methyl |
| 10 | Al | Me | 1 | H | Me | Adamantyl | H | Methyl |
| 11 | Al | Me | 1 | H | Cl | Cl | H | Methyl |
| 12 | Al | —OBn (—O—CH₂Ph) | 1 | H | H | H | H | Methyl |
| 13 | Al | —OBn (—O—CH₂Ph) | 1 | H | t-Butyl | t-Butyl | H | Methyl |
| 14 | Al | —OBn (—O—CH₂Ph) | 1 | H | OMe | t-Butyl | H | Methyl |
| 15 | Al | —OTol (—O—4—C₆H₄Me) | 1 | H | H | H | H | Methyl |
| 16 | Al | —OTol (—O—4—C₆H₄Me) | 1 | H | t-Butyl | t-Butyl | H | Methyl |
| 17 | Al | —OTol (—O—4—C₆H₄Me) | 1 | H | OMe | t-Butyl | H | Methyl |
| 18 | Al | Me | 1 | H | Naphthy fused to | | | H |
| | | | | | phenyl ring | | | |
| 19 | Al | —OBn (—O—CH₂Ph) | 1 | H | Naphthy fused to | | | Methyl |
| | | | | | phenyl ring | | | |
| 20 | Ti | OᵢPr | 2 | H | H | H | H | Methyl |
| 21 | Ti | OᵢPr | 2 | H | t-Butyl | t-Butyl | H | Methyl |
| 22 | Ti | OᵢPr | 2 | H | Naphthy fused to | | | Methyl |
| | | | | | phenyl ring | | | |
| 23 | Ti | OᵢPr | 2 | H | OMe | t-Butyl | H | Methyl |
| 24 | Ti | OᵢPr | 2 | H | Me | Adamantyl | H | Methyl |
| 25 | Ti | OᵢPr | 2 | H | Cl | Cl | H | Methyl |
| 26 | Ti | OᵢPr | 2 | H | Me | Adamantyl | H | Methyl |
| 27 | Ti | OᵢPr | 2 | Me | H | H | H | Methyl |
| 28 | Ti | Cl | 2 | H | OMe | t-Butyl | H | Methyl |
| 29 | Cr | Cl | 1 | H | H | H | H | Methyl |
| 30 | Cr | Cl | 1 | H | t-Butyl | t-Butyl | H | Methyl |
| 31 | Co | Cl | 1 | H | H | H | H | Methyl |
| 32 | Co | Cl | 1 | H | t-Butyl | t-Butyl | H | Methyl |
| 33 | Zn | — | 0 | H | H | H | H | Methyl |
| 34 | Al | OBn | 1 | Me | H | H | OMe | Methyl |

| | | | | | | |
|---|---|---|---|---|---|---|
| 35 | Al | OTol | 1 | H | Naphthy fused to | H |
| | | | | | phenyl ring | |

Uses of the Compounds and Ligands

The presently disclosed compounds and ligands may, for example, be used as catalysts in various reactions.

Co-Polymerisation of Anhydrides and Epoxides

The presently disclosed compounds and ligands may, for example, be used for the co-polymerisation, for example the ring-opening co-polymerisation (ROCOP), for example the co-polymerisation or the ring-opening co-polymerisation of anhydrides and epoxides, for example cyclic anhydrides and epoxides. Surprisingly, it has been discovered that the ester product can be obtained in very high yields when the presently disclosed compounds are present during the ROCOP reaction, as well as producing highly alternating co-polymers for a range of both anhydrides and epoxides.

The epoxide and anhydride may be any epoxide or anhydride suitable to undergo a ring-opening co-polymerization reaction.

Suitable anhydride starting materials include, for example, succinic anhydride, phthalic anhydride, tetrafluorophthalic anhydride, tetrachlorophthalic anahydride, tetrabromophthalic anhydride, citraconic anhydride, itaconic anhydride, cis-1,2-cyclohexanedicarboxylic anhydride, cis-1,2,3,6-tetrahydrophthalic anhydride, 2,3-dichloromaleic anhydride, 1,2,4,5-benzenetetracarboxylic anhydride, and maleic anhydride.

Suitable epoxide starting materials include, for example, styrene oxide, cyclohexene oxide, epichlorohydrin, isobutylene oxide, propylene oxide, 3,4-epoxy-1-butene and limonene oxide

The reaction may also use a co-catalyst. Suitable co-catalysts include, for example, bis(triphenylphosphoranylidene)ammonium chloride, tetrabutylammonium chloride, and tetrabutylammonium bromide. The presently disclosed L-M-Xₙ compounds may be referred to as a “pre-catalyst” and may react with the co-catalyst to give a (non-isolated) catalytically active species.

The reaction may or may not take place in the presence of a solvent, for example a polar or non-polar solvent. Suitable solvents include, for example, toluene and tetrahydrofuran (THF).

The reaction may take place at any suitable temperature and pressure. The temperature may, for example, range from about 20° C. to about 200° C., for example from about 30° C. to about 180° C., for example from about 40° C. to about 160° C., for example from about 50° C. to about 150° C., for example from about 60° C. to about 140° C., for example from about 70° C. to about 130° C., for example from about 80° C. to about 120° C. The pressure may, for example, range from about 50 kPa to about 150 kPa, for example from about 60 kPa to about 140 kPa, for example from about 70 kPa to about 130 kPa, for example from about 80 kPa to about 120 kPa, for example from about 90 kPa to about 110 kPa, for example from about 95 kPa to about 105 kPa.

The reaction may take place for a period of time ranging from about 30 minutes to about 48 hours. For example, the reaction may take place for a period of time ranging from about 1 hour to about 36 hours or from about 6 hours to about 36 hours or from about 12 hours to about 36 hours or from about 12 hours to about 24 hours.

In some embodiments, the reaction mole ratios may any one of more of the following:

| | Possible | Possible | Possible | Possible | Possible |
|---|---|---|---|---|---|
| | mole ratio | mole ratio | mole ratio | mole ratio | mole ratio |
| Compound/Pre- | about 100:1 | about 50:1 | about 20:1 | about 5:1 | about 1:1 |
| catalyst:Co- | to about | to about | to about | to about | |
| catalyst | 1:100 | 1:50 | 1:20 | 1:5 | |
| Compound/Pre- | about 1:1 | about 1:10 | about 1:50 | about 1:100 | about 1:200 |
| catalyst:Epoxide | to about | to about | to about | to about | to about |
| | 1:100,000 | 1:10,000 | 1:5000 | 1:2000 | 1:1000 |
| Compound/Pre- | about 1:1 | about 1:10 | about 1:50 | about 1:75 | about 1:100 |
| catalyst:Anhydride | to about | to about | to about | to about | to about |
| | 1:5000 | 1:1000 | 1:750 | 1:500 | 1:300 |
| Co- | about 1:1 | about 1:10 | about 1:50 | about 1:100 | about 1:200 |
| catalyst:Epoxide | to about | to about | to about | to about | to about |
| | 1:100,000 | 1:10,000 | 1:5000 | 1:2000 | 1:1000 |
| Co- | about 1:1 | about 1:10 | about 1:50 | about 1:75 | about 1:100 |
| catalyst:Anhydride | to about | to about | to about | to about | to about |
| | 1:5000 | 1:1000 | 1:750 | 1:500 | 1:300 |
| Epoxide:Anhydride | about 20:1 | about 15:1 | about 10:1 | about 5:1 | about 5:1 |
| | to about | to about | to about | to about | to about |
| | 1:20 | 1:15 | 1:1 | 1:5 | 1:1 |

The catalyst loading level may, for example, range from about 0.01 mol % to about 2 mol %, for example from about 0.05 mol % to about 1 mol %, for example from about 0.1 mol % to about 0.5 mol %, for example from about 0.1 mol % to about 0.3 mol %, for example about 0.2 mol %. This is based on moles of catalyst to moles of substrate.

After the ring-opening co-polymerisation has completed, the polymer product may be retried by any suitable method. For example, the polymer may be precipitated, for example using an alcohol such as methanol or ethanol.

ROP of Cyclic Esters

The presently disclosed compounds and ligands may, for example, be used for ring-opening polymerisation (ROP), for example the ring-opening polymerisation of cyclic esters to make polyesters. These polyesters may be used commercially as biodegradable polymers, with applications in food packaging and in the medical sector (e.g. suture). Surprisingly, it has been discovered that the product can be obtained in very high yields under mild conditions when the presently disclosed compounds are present during the ROP reaction.

The cyclic ester may be any cyclic ester suitable to undergo a ring-opening polymerization reaction.

Suitable cyclic ester starting materials include, for example ε-caprolactone and rac-lactide.

The reaction may also use a co-catalyst. Suitable co-catalysts include, for example alcohols such as phenols, benzyl alcohol and isopropyl alcohol.

The reaction may or may not take place in the presence of a solvent. Suitable solvents include, for example, toluene and tetrahydrofuran (THF).

The reaction may take place at any suitable temperature and pressure. The temperature may, for example, range from about 20° C. to about 200° C., for example from about 30° C. to about 180° C., for example from about 40° C. to about 160° C., for example from about 50° C. to about 150° C., for example from about 60° C. to about 140° C., for example from about 70° C. to about 130° C., for example from about 80° C. to about 120° C. The pressure may, for example, range from about 50 kPa to about 150 kPa, for example from about 60 kPa to about 140 kPa, for example from about 70 kPa to about 130 kPa, for example from about 80 kPa to about 120 kPa, for example from about 90 kPa to about 110 kPa, for example from about 95 kPa to about 105 kPa.

The reaction may take place for a period of time ranging from about 30 minutes to about 48 hours. For example, the reaction may take place for a period of time ranging from about 1 hour to about 36 hours or from about 6 hours to about 36 hours or from about 12 hours to about 36 hours or from about 12 hours to about 24 hours.

In some embodiments, the reaction mole ratios may any one of more of the following:

| | Possible | Possible | Possible | Possible | Possible |
|---|---|---|---|---|---|
| | mole ratio | mole ratio | mole ratio | mole ratio | mole ratio |
| Catalyst:Co- | about 10:1 | about 5:1 | about 3:1 | about 2:1 | about 1:0 |
| catalyst | to about | to about | to about | to about | |
| | 1:10 | 1:5 | 1:3 | 1:2 | |
| Catalyst:cyclic | about 1:1 | about 1:10 | about 1:20 | about 1:55 | about 1:75 |
| ester | to about | to about | to about | to about | to about |
| | 1:5000 | 1:1000 | 1:500 | 1:300 | 1:200 |
| Co- | about 1:1000 | about 1:1000 | about 1:500 | about 1:300 | about 1:200 |
| catalyst:cyclic | to about | to about | to about | to about | to about |
| ester | 1:1 | 1:10 | 1:20 | 1:30 | 1:50 |

The catalyst loading level may, for example, range from about 0.01 mol % to about 2 mol %, for example from about 0.05 mol % to about 1 mol %, for example from about 0.1 mol % to about 0.5 mol %, for example from about 0.1 mol % to about 0.3 mol %, for example about 0.2 mol %. This is based on moles of catalyst to moles of substrate.

After the ring-opening co-polymerisation has completed, the polymer product may be retried by any suitable method. For example, the polymer may be precipitated, for example using an alkane such as n-hexane or an alcohol, for example methanol or ethanol.

Trimerization of Isocyanates

Another possible use of the compounds and ligands of the invention are as catalysts in the trimerization of isocyanates under mild conditions.

Polyurethane foams have found widespread utility in the construction industry, particularly in insulation boards. Polyurethanes are prepared from diisocyanates and polyols, but cross-linking with isocyanurates is a crucial step in controlling their mechanical and thermosetting properties. Isocyanurates may be prepared by the cyclotrimerisation of the cyanate ion, followed by functionalization, for example, with alkyl halides. However a more versatile and atom-efficient approach is the trimerisation of isocyanates. Isocyanates are known to oligomerise in the presence of nucleophilic reagents, to afford (in addition to isocyanurates) uretdiones or iminooxazadiazinediones; in many cases a mixture of these products is obtained. As a specific example, the use of CsF will trimerise isocyanate at room temperature to give mixtures of products, including the corresponding isocyanurate, but is more selective at 150° C. Moreover, catalyst loading are typically high; in the above example, 5-10 mol % of catalyst is required.

Surprisingly, it has been found that compounds and ligands according to the present invention can selectively trimerise a wide range of isocyanates and isothiocyanates at room temperature with and with low catalyst loadings.

The isocyanate may be any isocyanate suitable to undergo a trimerization reaction. Isocyanates may have the structure R—N═C═O, where R is any suitable functional group. The isocyanate may, for example, be a di-isocyanate having the structure O═C═N—R—N═C═O. The isocyanate may, for example, be a tri-isocyanate or a tetra-isocyanate. In certain embodiments, isocyanates with more sterically demanding R groups may require higher temperatures than isocyanates with less sterically demanding R groups.

Suitable isocyanate starting materials include, for example, isocyanates (e.g. diisocyanates) wherein R is selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)2C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl

Suitable isocyanate starting materials include, for example, isocyanates (e.g. diisocyanates) wherein R is selected from optionally substituted C1-C8 linear straight chain or branched alkyl, optionally substituted C3-C15 cycloalkyl, optionally substituted C2-C8 alkenyl, optionally substituted C2-C8 alkynyl, phenyl and substituted phenyl, for example phenyl substituted with a C1-C8 alkyl group such as methyl.

The isocyanate starting materials may, for example, be dry before the trimerization reaction is performed. For example, the isocyanate starting materials may be dried by filtering through P₂O₅ before the trimerization reaction is performed.

The reaction may also use a co-catalyst. Suitable co-catalysts include, for example alcohols such as phenols, benzyl alcohol and isopropyl alcohol.

The reaction may or may not take place in the presence of a solvent. Suitable solvents include, for example, toluene and tetrahydrofuran (THF).

The reaction may take place at any suitable temperature and pressure. The temperature may, for example, range from about 20° C. to about 200° C., for example from about 30° C. to about 180° C., for example from about 40° C. to about 160° C., for example from about 50° C. to about 150° C., for example from about 60° C. to about 140° C., for example from about 70° C. to about 130° C., for example from about 80° C. to about 120° C. The pressure may, for example, range from about 50 kPa to about 150 kPa, for example from about 60 kPa to about 140 kPa, for example from about 70 kPa to about 130 kPa, for example from about 80 kPa to about 120 kPa, for example from about 90 kPa to about 110 kPa, for example from about 95 kPa to about 105 kPa.

The reaction may take place for a period of time ranging from about 30 minutes to about 48 hours. For example, the reaction may take place for a period of time ranging from about 1 hour to about 36 hours or from about 6 hours to about 36 hours or from about 12 hours to about 36 hours or from about 12 hours to about 24 hours. In certain embodiments, the reaction takes place for a period of time less than about 12 hours, for example less than about 8 hours, for example less than about 6 hours, for example less than about 4 hours, for example less than about 2 hours.

In some embodiments, the reaction mole ratios may any one of more of the following:

| | Possible | Possible | Possible | Possible | Possible |
|---|---|---|---|---|---|
| | mole ratio | mole ratio | mole ratio | mole ratio | mole ratio |
| Catalyst:Co- | about 10:1 | about 5:1 | about 3:1 | about 2:1 | about 1:0 |
| catalyst | to about | to about | to about | to about | |
| | 1:10 | 1:5 | 1:3 | 1:2 | |
| Catalyst:isocyanate | about 1:1 | about 1:10 | about 1:20 | about 1:50 | about 1:100 |
| | to about | to about | to about | to about | to about |
| | 1:5000 | 1:3000 | 1:2000 | 1:1000 | 1:500 |
| Co- | about 1:1 | about 1:10 | about 1:20 | about 1:50 | about 1:100 |
| catalyst:isocyanate | to about | to about | to about | to about | to about |
| | 1:5000 | 1:3000 | 1:2000 | 1:1000 | 1:500 |

Suitable catalyst loading levels for the trimerization reactions described herein may, for example, be from about 0.01 mol % to about 2 mol %, for example from about 0.05 mol % to about 1 mol %, for example from about 0.1 mol % to about 0.5 mol %, for example from about 0.1 mol % to about 0.3 mol %, for example about 0.2 mol %. This is based on moles of catalyst to moles of substrate (moles of catalyst/moles of substrate×100).

The product of the reaction may form a precipitate and be removed from the reaction media by any suitable method.

Methods of Making the Compounds and Ligands

Any suitable method may be used to synthesize the compounds and ligands disclosed herein, as would be understood by the person skilled in the art.

One such method is through the Schiff base condensation of pyridyl-diamine compounds such as 2-pyridyl-1,3-propanediamine (ppda) with appropriate aldehydes or ketones, for example as shown in the scheme below. 2-pyridyl-1,3-propanediamine may, for example, be made from 2-ethylpyridine as described in Friedrich et al., Chem. Ber/Recueil, 1997, 130, 1751, the contents of which are incorporated herein by reference.

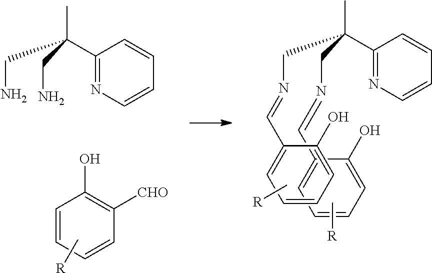

The choice of the aldehyde R groups will depend on the desired R groups of the final compound, as would be understood by the person skilled in the art.

Suitable reaction conditions for the Schiff base condensation reaction include combining the reactants at a temperature ranging from about 40° C. to about 100° C., for example from about 40° C. to about 80° C., for example from about 40° C. to about 60° C. For example, the chosen aldehyde and 2-pyridyl-1,3-propanediamine (ppda) may be reacted in methanol at 50° C. for 2 hours.

Any suitable solvent may be used, for example, methanol.

The protio-ligands formed from the reaction above are then further reacted in order to form the final compounds. The choice of reactant will depend on the desired final compound. In general, the reactant may be a metal complex where the metal is the metal that is desired in the final compound (M) and the ligands are organometallic ligands such as alkyl groups (e.g. methyl or ethyl), halogen groups (e.g. chloride or bromide), THF, water or alkoxy groups (e.g. methoxy, ethoxy or propoxy). One or more of the organometallic ligands may correspond to X in the final compound.

For example, if it is desired to have M=Al, then reaction with an organometallic precursor, for example, AlXY₂ (Y=organometallic ligand, for example methyl or ethyl; X=organometallic ligand or halide ligand, for example methyl, ethyl, chloride, bromide) may be performed.

For example, if it is desired to have M=Al and X=Cl, then reaction with AlClEt₂, for example, in toluene, may be performed.

For example, if it is desired to have M=Al and X=Me, then a reaction with AlMe₃, for example, in toluene at, for example, room temperature may be performed.

Such compounds may be further reacted with an alcohol (ROH) to form the respective aryloxide or alkoxide compounds with X=OR.

Suitable reaction conditions for this step include combining the reactants at a temperature ranging from about 10° C. to about 100° C., for example from about 40° C. to about 80° C., for example from about 40° C. to about 60° C.

For example, the reaction of the protio-ligand with the aluminium precursor may take place in toluene for 2 hours at 20° C. or 80° C.

For example, if it is desired to have M=Ti, X=OⁱPr, and n=2, then reaction with Ti(OⁱPr) may be performed.

For example, if it is desired to have M=Ti, X=Cl and n=1, then reaction with KH followed by TiCl₃(THF)₃ may be performed.

For example, if it is desired to have M=Cr, X=Cl, then reaction with KH followed by CrCl₃(THF)₃ may be performed in accordance with the procedure reported by Darenbourg et al., Inorg. Chem., 2004, 43, 6024, the contents of which are incorporated herein by reference.

For example, if it is desired to have M=Co, X=Cl, then reaction with Co(O₂CCH₃)₂(H₂O)₄ may be performed in accordance with the procedure reported by Coates et al., Dalton. Trans., 2006, 237, the contents of which are incorporated herein by reference.

For example, if it is desired to have M=Zn, then reaction with an organometallic precursor, for example ZnMe₂ or ZnEt₂, may be performed.

The diamine precursor (e.g. ppda) can be prepared using the synthetic procedure reported for the diamido-pyridine ligands, for example in Friedrich et al., Chem. Ber./Recueil, 1997, 130, 1751, Shakya et al., Inorg. Chem., 2011, 50, 11581 and Shakya et al., Eur. J. Inorg. Chem., 2009, 5319, the contents of which are incorporated herein by reference.

The intermediate compounds in these reactions are also disclosed herein and are part of the present invention. The intermediate compounds may have functional groups in accordance with the variables M, X, n, R1, R2, R3, R4, R5, R6, R7, R8 and R9 described above for the ligands and catalysts described herein.

### EXAMPLES

### Example 1—Synthesis of Catalysts

Protio-ligands (L of Formula (I)) were prepared by a Schiff base condensation between 2-methyl-2-pyridin-2-yl-propane-1,3-diamine (ppda) and an optionally substituted salicylaldehyde as shown in the reaction scheme below.

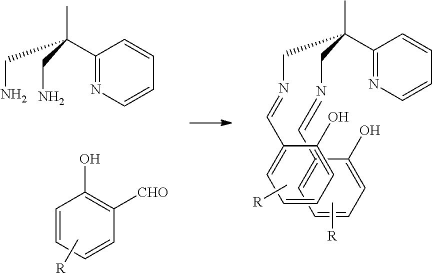

Many of the aldehyde/ketone starting materials were obtained from commercial sources (Fisher Scientific, Acros Organics, TCl Chemicals). Some examples were not commercially available, and were prepared using published procedures. For examples, 3-tert-butyl-5-methoxysalicylaldehyde, and 3-adamantyl-5-methylsalicylaldehyde were prepared by the procedures described in Kurahashi and Fuji, J. Am. Chem. Soc., 2011, 133, 8307 and Sattler et al., Organometallics, 2013, 32, 6899.

The reaction conditions for the Schiff base condensation reaction were as follows: A solution of the chosen aldehyde (18.5 mmol) in methanol (15 mL) was added to a stirred solution of 2-pyridyl-1,3-propanediamine (ppda) (1.50 g, 9.07 mmol) in methanol (15 mL). The resulting yellow solution was stirred at 50° C. for 2 hours. The pale-yellow solution was then allowed to cool to room temperature, whereupon a yellow precipitate formed, which was filtered and washed with cold methanol. The product was dried in vacuo for several hours. Typical yield: 85-90%. The reaction with 2-hydroxy-1-naphthaldehyde was undertaken under an argon atmosphere with recrystallised aldehyde.

The protio-ligands (H₂L) were then reacted with a metal complex in toluene to form compounds of the general formula L-M-Xₙ where X is Me or Cl. The metal complex was AlMe₃ where M is Al and X is Me.

For M=Al and X=Me, the reaction conditions were as follows: AlMe₃ 1.83 mL (2.0 M in toluene, 3.6 mmol) was added dropwise to a stirred solution of H₂L (3.6 mmol) in toluene (20 mL). The reaction mixture was heated at 80° C. for 18 hours (for L derived from salicylaldehyde) or at 20° C. for 18 hours for all other L derivatives, after which the solvent was removed under reduced pressure. The residue was washed with hexanes (2×15 mL) to afford an off-white solid. Typical yield: 1 (80-90%).

For M=Al and X=OR, compounds having X as Me were further reacted with an alcohol (ROH). The reaction conditions were as follows: A solution of dry benzyl alcohol (0.3 mL, 2.9 mmol) in toluene (10 mL) was added to a solution of [Al(L)Me] (2.87 mmol) in toluene (30 mL), and the solution was stirred at room temperature for 24 h. The solvent was concentrated to half the original volume to afford a precipitate, which was filtered, and washed with hexanes (2×20 mL) to give an off-white solid. Typical yield: (80-90%).

For M=Ti, X=Cl, and n=1, the reaction conditions were as follows: A solution of L (3.6 mmol) in THF was added dropwise to a solution of TiCl₃(THF)₃ (3.6 mmol) in THF at room temperature. The reaction mixture was stirred for 30 min, upon which a precipitate formed. The solid was isolated by cannula filtration and washed with dry diethyl ether (3×10 ml). The solid complex was dried in vacuo, typical yield: 70-80%.

For M=Ti, X=OⁱPr, and n=2, the reaction conditions were as follows: To a solution of L (3 mmol) in dichloromethane (10 mL), was added a solution of Ti(OⁱPr)₄ (0.85 g, 3 mmol) in dichloromethane (5 mL). The resulting solution was stirred at room temperature for 4 hours, after which the volatiles were removed under reduced pressure. The residue was washed with cold pentane (2×5 mL) and dried in vacuo to obtain the titled complex as a pale yellow solid. Typical yield: 80-90%.

For M=Cr and X=Cl, the reaction conditions were as folllows: In an inert atmosphere, KH (130 mg, 3.18 mmol) and L (1.51 mmol) were suspended in THF (40 mL) and stirred overnight at room temperature. The reaction was filtered via cannula into a suspension of CrCl₃(THF)₃ (620 mg, 1.66 mmol) in THF (10 ML) and stirred at room temperature for 24 hours. Solvent was removed and the resulting brown solid was dissolved in dichloromethane. The solution was filtered to remove KCl, and the volatiles removed under reduced pressure, affording a brown solid. Typical yield: 60-70%.

For M=Co and X=Cl, the reaction conditions were as follows: Cobalt acetate tetrahydrate (1.00 g, 4.02 mmol) and L (3.35 mmol) were dissolved in methanol (40 mL) and toluene (40 mL) under an argon atmosphere, and the reaction stirred for 2 hours at room temperature. The solvent was removed under reduced pressure, producing a brown precipitate, which was used without further purification. The intermediate, p-toluene sulfonic acid monohydrate (0.637 g, 3.35 mmol) and dichloromethane (100 mL) were added to a round bottom flask under air and stirred at room temperature for 2 hours. The solvent was removed under reduced pressure, and the product washed with petroleum ether and subsequently dissolved in dichloromethane. The solution was washed with a saturated solution of NaCl the solvent was removed under reduced pressure to afford a dark green precipitate. The precipitate was dissolved in ethanol, filtered, and the volatiles removed under reduced pressure to produce the final product. Typical yield: 60-70%.

For M=Zn, the reaction conditions were as follows: To a solution of L (3 mmol) in toluene (10 mL) was added a solution of ZnEt₂ (3.3 mL of a 0.9 M solution in hexanes, 3 mmol) at room temperature, and the reaction stirred for 2 hours. The volatiles were removed under reduced pressure to yield the title complex. Typical yield: 80-90%.

The compounds shown in the table below were synthesised.

| | | | | R1, | | | R8, | |
|---|---|---|---|---|---|---|---|---|
| Compound | M | X | n | R2 | R3, R6 | R4, R5 | R9 | R7 |
| 1 | Al | Cl | 1 | H | H | H | H | Methyl |
| 2 | Al | Cl | 1 | H | t-Butyl | t-Butyl | H | Methyl |
| 3 | Al | Cl | 1 | H | OMe | t-Butyl | H | Methyl |
| 4 | Al | Cl | 1 | H | Me | Adamantyl | H | Methyl |
| 5 | Al | Me | 1 | H | H | H | H | Methyl |
| 6 | Al | —OTol (—O—4—C₆H₄Me) | 1 | H | H | H | H | Methyl |
| 7 | Al | —OBn (—O—CH₂Ph) | 1 | H | H | H | H | Methyl |
| 8 | Al | Me | 1 | H | t-Butyl | t-Butyl | H | Methyl |
| 9 | Al | Me | 1 | H | OMe | t-Butyl | H | Methyl |
| 10 | Al | Me | 1 | H | Me | Adamantyl | H | Methyl |
| 11 | Al | Me | 1 | H | Cl | Cl | H | Methyl |
| 12 | Al | —OBn (—O—CH₂Ph) | 1 | H | H | H | H | Methyl |
| 13 | Al | —OBn (—O—CH₂Ph) | 1 | H | t-Butyl | t-Butyl | H | Methyl |
| 14 | Al | —OBn (—O—CH₂Ph) | 1 | H | OMe | t-Butyl | H | Methyl |
| 15 | Al | —OTol (—O—4—C₆H₄Me) | 1 | H | H | H | H | Methyl |
| 16 | Al | —OTol (—O—4—C₆H₄Me) | 1 | H | t-Butyl | t-Butyl | H | Methyl |
| 17 | Al | —OTol (—O—4—C₆H₄Me) | 1 | H | OMe | t-Butyl | H | Methyl |
| 18 | Al | Me | 1 | H | Naphthy fused to | | | H |
| | | | | | phenyl ring | | | |
| 19 | Al | —OBn (—O—CH₂Ph) | 1 | H | Naphthy fused to | | | Methyl |
| | | | | | phenyl ring | | | |
| 20 | Ti | OᵢPr | 2 | H | H | H | H | Methyl |
| 21 | Ti | OᵢPr | 2 | H | t-Butyl | t-Butyl | H | Methyl |
| 22 | Ti | OᵢPr | 2 | H | Naphthy fused to | | | Methyl |
| | | | | | phenyl ring | | | |
| 23 | Ti | OᵢPr | 2 | H | OMe | t-Butyl | H | Methyl |
| 24 | Ti | OᵢPr | 2 | H | Me | Adamantyl | H | Methyl |
| 25 | Ti | OᵢPr | 2 | H | Cl | Cl | H | Methyl |
| 26 | Ti | OᵢPr | 2 | H | Me | Adamantyl | H | Methyl |
| 27 | Ti | OᵢPr | 2 | Me | H | H | H | Methyl |
| 28 | Ti | Cl | 2 | H | OMe | t-Butyl | H | Methyl |
| 29 | Cr | Cl | 1 | H | H | H | H | Methyl |
| 30 | Cr | Cl | 1 | H | t-Butyl | t-Butyl | H | Methyl |
| 31 | Co | Cl | 1 | H | H | H | H | Methyl |
| 32 | Co | Cl | 1 | H | t-Butyl | t-Butyl | H | Methyl |
| 33 | Zn | — | 0 | H | H | H | H | Methyl |
| 34 | Al | OBn | 1 | Me | H | H | OMe | Methyl |

| | | | | | | |
|---|---|---|---|---|---|---|
| 35 | Al | OTol | 1 | H | Naphthy fused to | H |
| | | | | | phenyl ring | |

The compounds were all analysed by one or more of ¹H and ¹³C{¹H} NMR spectroscopy, elemental combustion analysis, infrared spectroscopy, mass spectrometry, and X-ray crystallography to confirm their identities.

NMR data of many of the prepared complexes indicated the presence of two species. Variable temperature ¹H NMR spectroscopy and X-ray crystallography enabled the identity of these species to be elucidated; one species corresponds to that with the pyridyl group coordinated to the metal centre and the other with the pyridyl being pendant (not coordinated). This means that the ligand L can exist in either κ⁵ or κ⁴ coordination modes. It was found that these two components exist in equilibrium with each other, and are not independently isolable. Density functional theory was used to investigate the activity of these compounds in the reactions described below and it was found that the pyridyl hemi-lability is an integral part of the reaction mechanisms, with L switching between the κ⁵ or κ⁴ coordination modes at various parts of the catalytic cycle.

### Example 2—Trimerization of Isocyanates

Compound 7 described in Example 1 above was tested as a catalyst in the selective trimerization of isocyanates to provide isocyanurates, using mild conditions. Surprisingly, Compound 7 was found to be a highly active catalyst in this reaction, with catalyst loadings down to 0.2 mol %.

In a nitrogen-filled glove box, Compound 7 was added to a 5 mL screw-cap vial and isocyanate was added via a measuring pipette. The vial was sealed, and stirred for the appropriate time (see Table 2 for details). For experiments that required heating, the vial was placed in a 20-well thermostat-controlled aluminium heating block, and heated for the appropriate amount of time with continuous stirring. Temperature was controlled by a thermocouple inserted into a “blank” reaction vial containing 2 mL of paraffin oil. After this time, a precipitate had formed, or for those experiments with high conversions, the reaction completely solidified. The isolated solids were characterized by ¹H NMR and IR spectroscopies which showed that no isocyanate remained; mass spectra exhibited ions only for the cyclotrimers.

This reaction with 0.2 mol % of compound led to quantitative conversion of phenyl isocyanate to N,N′,N″-triphenyl isocyanurate,

Recrystallization of the reaction product from CH₂C₂ gave a large quantity of crystals suitable for single crystal X-ray diffraction; the structure was thereby confirmed and is shown below.

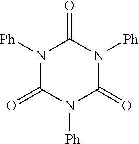 [SMILES: O=c1n(-c2ccccc2)c(=O)n(-c2ccccc2)c(=O)n1-c1ccccc1]

Various reaction conditions using Compound 7 and an excess of phenyl isocyanate were tested and it was discovered that the reaction will operate at catalyst loadings down to 0.2 mol %, giving quantitative conversion to the isocyanurate overnight at 20° C. or in 1 hour at 50° C. The bulk sample was analysed by powder X-ray diffraction and it was shown that the cyclotrimer is the only detectable crystalline material in the sample. In addition, analysis of the ¹H, ¹³C{¹H} and ¹⁵N NMR spectra of the crude reaction mixture provides no evidence for any other species other than the trimer (such as dimers or polymer). It can therefore be concluded that Compound 7 is highly selective for this transformation, which contrasts with many other catalytic systems that can often give rise to various side products in addition to the isocyanurate.

Compound 7 was further tested using a range of different isocyanates. The trimerization reaction was tested with ethyl, 4-tolyl, benzyl, allyl, and tert-butyl isocyanates, and 1,4-phenyl and 2,4-tolyl diisocyanates. In each case, the cyclotrimer was obtained, and their identity verified using IR and NMR spectroscopies, and mass spectrometry. In no case were any oligomers, other than the trimer, detected. This shows that Compound 7 is a versatile catalyst for this transformation.

The results of the reactions are shown in the Table below.

| Isocyanate | Temp ° C. | Time | | Yield % |
|---|---|---|---|---|
| 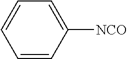 | 25 | 18 | h | 98 |
| 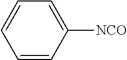 | 50 | 1 | h | 95 |
| 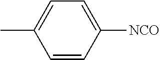 | 25 | 48 | h | 25 |
| 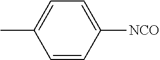 | 50 | 18 | h | 98 |
| 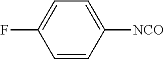 | 25 | 12 | | 93 |
| 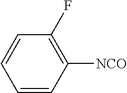 | 25 | 48 | | 95 |
| 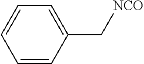 | 25 | 3 | h | 77 |
| 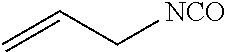 | 25 | 3 | h | 59 |
| 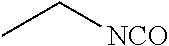 | 25 | 30 | min. | 98 |
| 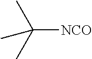 | 25 | 2 | h | 5 |
| 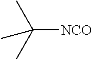 | 50 | 18 | h | 96 |
| 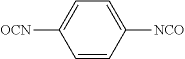 | 100 | 4 | h | 71 |
| 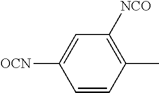 | 25 | 90 | min. | 89 |
| 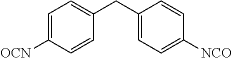 | 50 | 48 | | 90 |

### Example 3—Ring-Opening Co-Polymerization of Cyclic Anhydrides and Epoxides

Compounds 1, 2 and 3, described in Example 1 above were tested in the ring-opening co-polymerisation of cyclic anhydrides and epoxides.

Compound A was used as a control. In compound A, M was Al, X was Cl, n was 1 and L had the following structure.

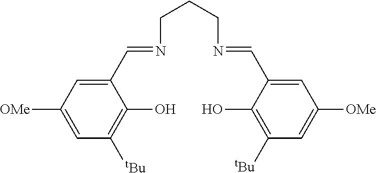 [SMILES: COc1cc(/C=N/CCC/N=C/c2cc(OC)cc(C(C)(C)C)c2O)c(O)c(C(C)(C)C)c1]

In a glove box, a 5 mL screw-cap vial was charged with one of compounds 1, 2 or 3 (7×10⁻³ mmol, ca. 3 mg), bis(triphenylphosphine)iminium chloride (7×10⁻³ mmol, 1 equiv.), and 1.5 mL of toluene. To this was added the appropriate amounts of anhydride and epoxide (1.4 mmol of each). The vial was sealed and removed from the glove box. The vial was placed in a 20-well thermostat-controlled aluminium heating block, and heated for the appropriate amount of time with continuous stirring. Temperature was controlled by a thermocouple inserted into a “blank” reaction vial containing 2 mL of paraffin oil. After heating, the reaction was dissolved in dichloromethane and the polymer precipitated with methanol.

The results are shown in the Table below.

| | | | | | T | t | | | % |
|---|---|---|---|---|---|---|---|---|---|
| Compound | ratios^{a} | Epoxide^{b} | Anhydride^{c} | solvent | (° C.) | (h) | Mn | PDI | ester |
| 1 | 1:1:200:200 | CHO | PhA | Toluene | 70 | 24 | 4287 | 1.04 | 92 |
| 1 | 1:1:200:200 | ECH | PhA | Toluene | 70 | 24 | 6800 | 9.70 | 49 |
| 1 | 1:1:200:200 | PO | PhA | Toluene | 70 | 24 | 2269 | 1.22 | 91 |
| 1 | 1:1:200:200 | ECH | TCPA | THF | 70 | 24 | 3494 | 1.45 | 42 |
| 1 | 1:1:200:200 | CHO | SA | Toluene | 70 | 24 | 1643 | 1.21 | 100 |
| 1 | 1:1:200:200 | ECH | SA | Toluene | 70 | 24 | 3239 | 1.62 | 50 |
| 1 | 1:1:200:200 | PO | SA | Toluene | 70 | 24 | 1893 | 1.24 | 34 |
| 1 | 1:1:200:200 | SO | SA | Toluene | 70 | 24 | 1910 | 1.29 | 95 |
| 1 | 1:1:200:200 | SO | PhA | Toluene | 70 | 24 | 2039 | 1.24 | 95 |
| 1 | 1:1:200:200 | LO | PhA | Toluene | 70 | 24 | 2589 | 1.99 | 75 |
| 1 | 1:1:200:200 | CHO | TCPA | Toluene | 70 | 24 | 5054 | 1.42 | 94 |
| 1 | 1:1:200:200 | PO | TCPA | Toluene | 70 | 24 | 4668 | 1.40 | 34 |
| 1 | 1:1:200:200 | SO | TCPA | Toluene | 70 | 24 | 571 | 1.38 | 92 |
| 1 | 1:1:200:200 | LO | TCPA | Toluene | 70 | 24 | 6070 | 2.10 | 85 |
| 2 | 1:1:200:200 | CHO | PhA | Toluene | 70 | 24 | 8008 | 1.03 | 94 |
| 3 | 1:1:200:200 | CHO | PhA | Toluene | 70 | 24 | 1655 | 1.38 | 84 |
| A | 1:1:200:200 | CHO | PhA | Toluene | 70 | 24 | 9491 | 1.03 | 96 |

^{a}compound:cocatalyst:epoxide:anhydride;
^{b}SO = styrene oxide, CHO = cyclohexene oxide, ECH epichlorohydrin, LO = limonene oxide, PO = propylene oxide;
^{c}SA = succinic anhydride, MA = maleic anhydride, PhA = phthalic anhydride, TCPA = tetrachlorophthalic anhydride.

### Example 4—Ring-Opening Polymerization of ε-Caprolactone

The chemicals used in the ε-CL polymerization reactions were weighed in a nitrogen-filled glove box. In general, polymerization reactions were carried out in toluene (3 ml) or (where specified) without solvent, at a prescribed temperature. The ratio of the monomer:catalyst:cocatalyst was 100:1:0 or 100:1:1 when solvent was used, or 400:1:0 or 400:1:1 when no solvent was used. The co-catalyst was either benzyl or isopropyl alcohol (0.015 mmol, 1 molar equivalent relative to the catalyst complex) where required. After stirring for 5 min, ε-caprolactone (1.5 mmol) was added. Polymerization experiments were conducted in a screw-cap vial that was removed from the glove box only after the reaction was fully assembled and the vial sealed. The reaction mixture was stirred at the prescribed temperature for the required time. The conversion was determined by ¹H NMR spectroscopy. The reaction was quenched by the addition of aqueous acetic acid (0.35 N, 10 mL). The resulting mixture was poured into 25 mL of hexanes to precipitate the polymer. Crude products were recrystallized from THF/hexane and dried in vacuo to a constant weight.

Compounds 5, 8 and 9 were initially tested in the ring-opening polymerisation of ε-caprolactone. The results are provided in the Table below.

| | | | T | t | Mₙ | Mₙ | Conv. | |
|---|---|---|---|---|---|---|---|---|
| Run | Compound | [M]₀:[Al]₀:[BnOH] | (° C.) | (h) | (obsd) | (calcd) | (%) | M_{w}/Mₙ |
| 1 | 5 | 100:1:0 | 25 | 2 | nd | — | 1 | nd |
| 2 | 5 | 100:1:0 | 25 | 3 | nd | — | 5 | nd |
| 3 | 5 | 100:1:0 | 25 | 4 | nd | — | 25 | nd |
| 4 | 8 | 100:1:0 | 25 | 1 | nd | — | 12 | nd |
| 5 | 8 | 100:1:0 | 25 | 2 | nd | — | 22 | nd |
| 6 | 8 | 100:1:0 | 25 | 3 | 26070 | 3840 | 33 | 1.08 |
| 7 | 8 | 100:1:0 | 25 | 4 | 32660 | 5096 | 44 | 1.02 |
| 8 | 9 | 100:1:0 | 25 | 1 | 15090 | 2214 | 19 | 1.01 |
| 9 | 9 | 100:1:0 | 25 | 2 | 19390 | 3618 | 32 | 1.00 |
| 10 | 9 | 100:1:0 | 25 | 3 | 29850 | 4622 | 41 | 1.00 |
| 11 | 5 | 100:1:0 | 50 | 1 | 22360 | 5250 | 47 | 1.15 |
| 12 | 5 | 100:1:0 | 50 | 2 | 31700 | 9353 | 81 | 1.01 |
| 13 | 5 | 100:1:0 | 50 | 3 | 31900 | 10951 | 95 | 1.02 |
| 14 | 5 | 100:1:0 | 50 | 4 | 33470 | 11345 | 99 | 1.02 |

It is unusual for complexes, especially aluminium complexes, to effect this reaction under ambient conditions. It is therefore surprising that the reaction was able to proceed at 25° C. using compounds 5, 8 and 9. A dramatic change was observed when a higher temperature was used, even without using a co-catalyst. An approximately total conversion of the monomer to polymer was obtained after 4 hours, with high molecular weight polymer produced with narrow polydispersity index (PDI) of 1.02, consistent with a highly controlled polymerization process with few side-reactions (termination events such as transesterification and back-biting of the polymer chain), in Runs 11 to 14. The experimental molecular weights determined by gel permeation chromatography (GPO) (corrected using the appropriate Mark-Houwink parameters, due to the use of poly(styrene) standards) are 3-4 times higher than those calculated based upon conversions and catalyst:monomer ratios, indicating that only a part of the aluminium catalyst is active (about 34% for entry 14, assuming one polymer chain per active metal centre). This suggests that the rate of propagation is fast compared to the rate of initiation.

The complexes also exhibited efficient catalytic activity for polymerization of ε-caprolactone in the presence of benzyl alcohol (BnOH). The in situ formation of an alkoxide species (where OBn replaced Me as the X co-ligand) under the reaction conditions employed was confirmed via ¹H NMR spectroscopy for the 1:1 reaction of each complex with BnOH.

Compound B was used as a control. In compound B, M was Al, X was Me, n was 1 and L had the following structure where R3 to R9 were H, and R7 was methyl.

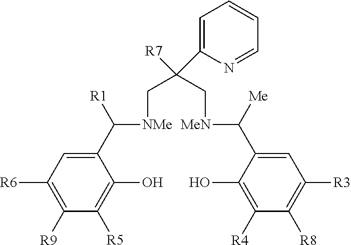

The results of the polymerization reactions of ε-caprolactone in the presence of benzyl alcohol are shown in the Table below.

| | | T | t | Mₙ | Mₙ | Conv. | |
|---|---|---|---|---|---|---|---|
| Compound | [M]₀:[Al]₀:[BnOH] | (° C.) | (h) | (obsd) | (calcd) | (%) | M_{w}/Mₙ |
| 5 | 100:1:1 | 25 | 1 | 4140 | 6842 | 59 | 1.09 |
| 5 | 100:1:1 | 25 | 2 | 5270 | 8500 | 74 | 1.07 |
| 5 | 100:1:1 | 25 | 3 | 8840 | 9354 | 82 | 1.06 |
| 5 | 100:1:1 | 25 | 4 | 9070 | 10838 | 94 | 1.04 |
| 8 | 100:1:1 | 25 | 1 | 8320 | 10097 | 88 | 1.02 |
| 8 | 100:1:1 | 25 | 2 | 8900 | 11186 | 98 | 1.02 |
| 8 | 100:1:1 | 25 | 3 | 9230 | 11300 | 99 | 1.01 |
| 8 | 100:1:1 | 25 | 4 | 9570 | 11500 | 100 | 1.01 |
| 18 | 100:1:1 | 25 | 1 | nd | n/a | 34 | nd |
| 18 | 100:1:1 | 25 | 2 | 4700 | 6950 | 61 | 1.0 |
| 18 | 100:1:1 | 25 | 3 | 6960 | 8332 | 73 | 1.10 |
| 18 | 100:1:1 | 25 | 4 | 10800 | 9240 | 83 | 1.05 |
| 18 | 100:1:1 | 25 | 6 | 12540 | 10946 | 94 | 1.05 |
| 9 | 100:1:1 | 25 | 0.5 | 7300 | 8100 | 70 | 1.02 |
| 9 | 100:1:1 | 25 | 1 | 11580 | 10000 | 87 | 1.01 |
| 9 | 100:1:1 | 25 | 1.5 | 13010 | 11236 | 98 | 1.01 |
| 9 | 100:1:1 | 25 | 2 | 13670 | 11500 | 100 | 1.00 |
| B | 100:1:1 | 25 | 1 | nd | n/a | 6 | nd |
| B | 100:1:1 | 25 | 2 | nd | n/a | 9 | nd |
| B | 100:1:1 | 25 | 3 | nd | n/a | 13 | nd |
| B | 100:1:1 | 25 | 4 | nd | n/a | 19 | nd |

Compound C was also used as a control. In compound C, M was Al, X was Me, n was 1 and L had the following structure.

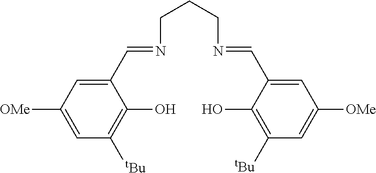 [SMILES: COc1cc(/C=N/CCC/N=C/c2cc(OC)cc(C(C)(C)C)c2O)c(O)c(C(C)(C)C)c1]

It was found that higher conversion was obtained with Compound 9 compared to 5 Compound C after 30 minutes.

Compound 8 also exhibited efficient catalytic activity for polymerization of ε-caprolactone in the presence of isopropanol.

The results of the polymerization reactions of ε-caprolactone in the presence of isopropanol are shown in the Table below.

| | | T | t | Mₙ | Mₙ | Conv. | |
|---|---|---|---|---|---|---|---|
| Compound | [M]₀:[Al]₀:[ⁱPrOH] | (° C.) | (h) | (obsd) | (calcd) | (%) | M_{w}/Mₙ |
| 8 | 100:1:1 | 25 | 1 | 8720 | 4054 | 35 | 1.18 |
| 8 | 100:1:1 | 25 | 2 | 14980 | 7821 | 68 | 1.13 |
| 8 | 100:1:1 | 25 | 3 | 16120 | 9761 | 85 | 1.12 |
| 8 | 100:1:1 | 25 | 4 | 18780 | 11245 | 98 | 1.10 |
| 8 | 100:1:1 | 50 | 1 | 13540 | 10446 | 91 | 1.18 |
| 8 | 100:1:1 | 50 | 2 | 14110 | 11245 | 98 | 1.19 |
| 8 | 100:1:1 | 50 | 3 | 14930 | 11359 | 99 | 1.27 |
| 8 | 100:1:1 | 50 | 4 | 15600 | 11474 | 100 | 1.22 |

### Example 5—Ring-Opening Polymerization of ε-Caprolactone

The same reaction described above in Example 4 was carried out at 25° C. using pre-prepared aluminium benzyloxy complexes. It is easier to manipulate aluminium alkoxide complexes since they are less sensitive to air/moisture and do not require a co-catalyst.

The results of the polymerization reaction are shown in the Table below.

| Compound | t (h) | Mn (obsd) | Mn (calcd) | Conv. (%) | Mw/Mn |
|---|---|---|---|---|---|
| 12 | 1 | 16040 | 9701 | 85 | 1.05 |
| 12 | 2 | 20140 | 11254 | 99 | 1.12 |
| 12 | 3 | 31400 | 11402 | 100 | 1.01 |
| 13 | 1 | 13520 | 9005 | 79 | 1.01 |
| 13 | 2 | 15090 | 10980 | 96 | 1.04 |
| 13 | 3 | 16890 | 11299 | 99 | 1.05 |
| 14 | 1 | 13810 | 6243 | 55 | 1.01 |
| 14 | 2 | 14660 | 9542 | 84 | 1.00 |
| 14 | 3 | 15960 | 10843 | 95 | 1.07 |
| 19 | 1 | 15630 | 5398 | 47 | 1.19 |
| 19 | 2 | 18870 | 8594 | 75 | 1.18 |
| 19 | 3 | 19040 | 10204 | 89 | 1.17 |
| 19 | 4 | 19280 | 11242 | 99 | 1.18 |
| 34 | 1 | n.d | — | 17 | — |
| 34 | 2 | 5690 | 4040 | 35 | 1.03 |
| 34 | 3 | 7770 | 6003 | 53 | 1.02 |
| 34 | 4 | 9050 | 7213 | 63 | 1.02 |
| 14^{†} | 4 | 41067 | 38459 | 84 | 1.13 |

^{†}Reaction run without added solvent as described above, using 400 equivalents of ε-caprolactone, where the ε-caprolactone acts as both the reacting monomer and solvent

It was found, for Compounds 12, 13, 14, 19 and 34 described in Example 1 above, that there is a first-order dependence of the rate of polymerization on monomer concentration. The rate of polymerization is shown in FIG. 1 below.

### Example 6—Ring-Opening Polymerization of ε-Caprolactone

The same reaction described above in Examples 4 and 5 was carried out using pre-prepared aluminium phenoxide complexes (where X is —Otol (—O-4-C₆H₄Me) at 80° C. These derivatives were found to be inactive at room temperature, therefore, the phenoxide complexes were tested at 80° C. for 4 hours.

The results of the polymerization reaction are shown in the Table below. Conversion was greater than 99% for all reactions.

| Compound | [M]₀:[Al]₀ | Mₙ (obsd) | Mₙ (calcd) | M_{w}/Mₙ |
|---|---|---|---|---|
| 15 | 50:1 | 5800 | 5650 | 1.04 |
| 15 | 100:1 | 6020 | 11300 | 1.04 |
| 15 | 150:1 | 6180 | 16950 | 1.03 |
| 15 | 200:1 | 6380 | 22600 | 1.04 |
| 15 | 250:1 | 6530 | 28250 | 1.04 |
| 16 | 50:1 | 5280 | 5650 | 1.02 |
| 16 | 100:1 | 5380 | 11300 | 1.02 |
| 16 | 150:1 | 5450 | 16950 | 1.02 |
| 16 | 200:1 | 5510 | 22600 | 1.02 |
| 16 | 250:1 | 5630 | 28250 | 1.02 |
| 35 | 50:1 | 5750 | 5650 | 1.03 |
| 35 | 100:1 | 5910 | 11300 | 1.04 |
| 35 | 150:1 | 6090 | 16950 | 1.03 |
| 35 | 200:1 | 6140 | 22600 | 1.03 |
| 35 | 250:1 | 6300 | 28250 | 1.03 |
| 17 | 50:1 | 5180 | 5650 | 1.02 |
| 17 | 100:1 | 5280 | 11300 | 1.02 |
| 17 | 150:1 | 5370 | 16950 | 1.02 |
| 17 | 200:1 | 5540 | 22600 | 1.02 |
| 17 | 250:1 | 5610 | 28250 | 1.02 |

### Example 7—Ring-Opening Polymerization of Rac-Lactide

The same reaction described above in Examples 4 to 6 was carried out using rac-lactide as the monomer instead of ε-caprolactone at a temperature of 80° C. The ratio of lactide:catalyst:co-catalyst was 100:1:1. The stereochemical microstructures of the PLA samples prepared were determined through inspection of the methine group of the homonuclear decoupled ¹H NMR spectra, giving the result “Pᵢ”.

The results are shown in the Table below.

| | t | Mₙ | Mₙ | Conv. | | |
|---|---|---|---|---|---|---|
| Compound | (h) | (obsd) | (calcd) | (%) | M_{w}/Mₙ | Pᵢ |
| 5 | 2 | 7810 | 12070 | 83 | 1.09 | 0.81 |
| 5 | 4 | 10450 | 13656 | 94 | 1.23 | 0.84 |
| 5 | 6 | 10600 | 13858 | 95 | 1.26 | 0.84 |
| 5 | 22 | 11020 | 13944 | 96 | 1.27 | 0.81 |
| 8 | 2 | 4380 | 7098 | 49 | 1.04 | 0.69 |
| 8 | 4 | 7110 | 10629 | 73 | 1.05 | 0.80 |
| 8 | 6 | 8520 | 12099 | 83 | 1.06 | 0.81 |
| 8 | 22 | 8970 | 14000 | 96 | 1.19 | 0.81 |
| 18 | 2 | <1250 | — | 86 | — | 0.74 |
| 18 | 4 | <1250 | — | 92 | — | 0.77 |
| 18 | 6 | <1250 | — | 94 | — | 0.79 |
| 18 | 22 | 7850 | — | 95 | 1.29 | 0.81 |
| 34 | 2 | <1250 | 7602 | 52 | — | — |
| 34 | 4 | <1250 | 10197 | 70 | — | 0.82 |
| 34 | 6 | 7800 | 10918 | 75 | 1.03 | 0.93 |
| 34 | 22 | 8880 | 13757 | 94 | 1.04 | 0.85 |
| 34 | 2 | 2700 | 3711 | 25 | 1.06 | — |
| 34 | 4 | 4460 | 5873 | 40 | 1.04 | 0.73 |
| 34 | 6 | 6390 | 8180 | 56 | 1.02 | 0.73 |
| 34 | 22 | 9050 | 12790 | 88 | 1.02 | — |

### Example 8—Ring-Opening Polymerization of Rac-Lactide

The same reaction described above in Example 7 was carried out using aluminium benzyloxy complexes described in Example 1 above. The results are shown in the Table below.

| | | Mₙ | Mₙ | Conv. | | |
|---|---|---|---|---|---|---|
| Compound | t (h) | (obsd) | (calcd) | (%) | M_{w}/Mₙ | Pᵢ |
| 12 | 2 | 12580 | 12294 | 85 | 1.13 | 0.86 |
| 12 | 4 | 15430 | 13173 | 91 | 1.13 | 0.81 |
| 12 | 6 | 16130 | 13807 | 96 | 1.24 | 0.86 |
| 13 | 4 | 8660 | 7206 | 50 | 1.03 | 0.84 |
| 13 | 6 | 12540 | 8863 | 62 | 1.02 | 0.87 |
| 13 | 22 | 19360 | 13303 | 92 | 1.01 | 0.92 |
| 19 | 1 | 13690 | 10293 | 71 | 1.08 | 0.72 |
| 19 | 2 | 18240 | 13159 | 91 | 1.23 | 0.73 |
| 19 | 3 | 18800 | 13259 | 92 | 1.23 | 0.69 |
| 19 | 4 | 20284 | 13951 | 97 | 1.28 | 0.67 |
| 14 | 2 | 7220 | 4900 | 34 | 1.03 | 0.88 |
| 14 | 4 | 11740 | 8143 | 57 | 1.02 | 0.82 |
| 14 | 6 | 15690 | 10449 | 73 | 1.01 | 0.90 |
| 14 | 22 | 27290 | 13750 | 95 | 1.00 | 0.86 |

The foregoing broadly describes certain embodiments of the present invention without limitation. Variations and modifications as will be readily apparent to those skilled in the art are intended to be within the scope of the present invention as defined in and by the appended claims.

1. A compound having the formula:
L-M-Xₙ
wherein:
M is a metal atom;
L is a ligand according to formula (I) 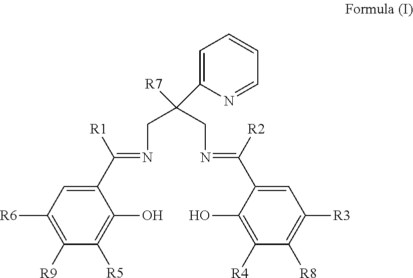
wherein,
R1 and R2 are the same or different and are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R—C(O)—, R′—C(O)—O—, R′—O—, R′₂N—C(O)—O—, R′(OR′)₂C—, (OR′)3C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC-(thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P-(phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3, R4, R5, R6, R8 and R9 are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8-alkyl) where V is selected from a single bond, O, S and NH, phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)-(amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC-(thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P-(phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R7 is hydrogen, optionally substituted C1-C8 linear straight chain or branched alkyl such as (CH2)_{y}OR′ and (CH2)_{y}NR′2, CN (nitrile) and R′sN— (amine), where each R′ is independently hydrogen, hydroxyl, halogen, optionally substituted C1-C8 alkoxyl, optionally substituted C1-C8 linear straight chain or branched alkyl, optionally substituted phenyl, optionally substituted C2-C8 alkenyl or optionally substituted C2-C8 alkynyl, and y is an integer from 1 to 8;
X is a co-ligand to balance the charge of the compound; and
n is an integer from 0 to 7;
excluding the compound where,
M is copper, X is methanol, n is 1, R1 to R6 are H, R7 is methyl, and R8 and R9 are H.

2. The compound of claim 1, wherein M is selected from scandium, titanium, chromium, manganese, iron, cobalt, nickel, copper, zinc, aluminium, gallium, yttrium, zirconium, niobium, molybdenum, technetium, ruthenium, rhodium, palladium, silver, cadmium, hafnium, tantalum, tungsten, rhenium, osmium, iridium, platinum, gold, thallium and lead.

3. The compound of claim 1, wherein M is selected from titanium, chromium, iron, cobalt, copper, aluminium and gallium.

4. The compound of claim 1, wherein the metal atom has an oxidation state of (II) or (III) or (IV).

5. The compound of claim 1, wherein X is selected from hydrogen, halogen, optionally substituted C1-C8 linear straight chain or branched alkyl, optionally substituted C1-C8 alkoxy, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, optionally substituted phenyl-V—(C1-C8-alkyl) where V is selected from a single bond, O, S and NH, optionally substituted phenyl-(C1-C8-alkyl)-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R″—C(O)—, R″—C(O)—O—, R″—O—, R″₂N—C(O)—O—, R″(OR″)₂C—, (OR″)3C—, (OR″)—C—O—, nitro (—NO2), nitrate (—ONO2), nitrile (CN), nitrite (—ONO), nitroso (NO), cyano (—OCN or —NCO), optionally substituted pyridyl, R″(NOH)C— (oxime), R″₃N-(amine), R″4N⁺— (quaternary amine), R″₂N—C(O)— (amide), R″(NR′)—C— (imine), R′(CO)NR″—C(O)— (imide), N₃ (azide), R″N₂— (azo), —SH (thiol), (OH)(O)S— (sulfino), (O)(O)(OH)S— (sulfo), thiocyanate (—SCN or —NCS), (S)HC— (thial), R′S— (sulfide), R′S—S— (disulphide), R″(O)S— (sulfoxide), (O)(O)R″S— (sulfonyl), R″SC (thioketone), R″S—C(O)— (thioester), R″C(O)—S—, R″₃P (phosphino), (OH)₂(O)P— (phosphono), (OH)₂(O)P—O— (phosphate) and (OR″)₂(O)P—O— (phosphoester), where each R″ is independently hydrogen, hydroxyl, halogen, optionally substituted C1-C8 alkoxy, optionally substituted C1-C8 linear straight chain or branched alkyl, optionally substituted C2-C8 alkenyl or optionally substituted C2-C8 alkynyl.

6. The compound of claim 1, where X is selected from hydrogen, halogen, optionally substituted C1-C8 linear straight chain or branched alkyl, optionally substituted C1-C8 alkoxy, optionally substituted C3-C15 cycloalkyl, optionally substituted phenyl, optionally substituted phenoxy, phenyl-V—(C1-C8)-alkyl where V is selected from a single bond, O, S and NH, phenyl-(C1-C8-alkyl)-V— where V is selected from a single bond, O, S and NH, and R″—O— where R″ is optionally substituted C1-C8 linear straight chain or branched alkyl.

7. The compound of claim 1, wherein X is selected from halogen, optionally substituted C1-C8 linear straight chain or branched alkyl, optionally substituted C1-C8 alkoxy, optionally substituted phenoxy, and phenyl-(C1-C8-alkyl)-V— where V is selected from a single bond, O, S and NH.

8. The compound of claim 1, wherein n is 0 or 1 or 2.

9. The compound of claim 1, wherein:
R1 and R2 are the same; and/or
R3 and R6 are the same; and/or
R4 and R5 are the same; and/or
R8 and R9 are the same; and/or
R3, R4, R5 and R6 are the same; and/or
R3, R4, R5, R6, R8 and R9 are the same.

10. The compound of claim 1, wherein R1 and R2 are hydrogen or optionally substituted C1-C8 linear straight chain or branched alkyl such as methyl.

11. The compound of claim 1, wherein R1 and R2 are hydrogen.

12. The compound of claim 1, wherein R8 and R9 are hydrogen or optionally substituted C1-C8 alkoxy such as methoxy.

13. The compound of claim 1, wherein R8 and R9 are hydrogen.

14. The compound of claim 1, wherein R3, R4, R5 and R6 are selected from hydrogen, halogen, optionally substituted C1-C8 alkoxy, optionally substituted C1-C8 linear straight chain or branched alkyl, optionally substituted phenyl which is optionally fused to the phenol ring in formula (I) to form a naphthalene moiety and optionally substituted C3-C15 cycloalkyl.

15. The compound of claim 1, wherein all of R3, R4, R5 and R6 are hydrogen.

16. The compound of claim 1, wherein all of R3, R4, R5 and R6 are a halogen such as chlorine.

17. The compound of claim 1, wherein one or both of R3 and R6 are an optionally substituted C1-C8 alkoxy such as methoxy.

18. The compound of claim 1, wherein one or both of R3 and R6 are an optionally substituted C1-C8 linear straight chain or branched alkyl such as butyl such as tertiary-butyl.

19. The compound of claim 1, wherein one or both of R3 and R6 are an optionally substituted C3-C15 cycloalkyl such as adamantyl.

20. The compound of claim 1, wherein one or both of R4 and R5 are an optionally substituted C1-C8 alkoxy such as methoxy.

21. The compound of claim 1, wherein one or both of R4 and R5 are an optionally substituted C1-C8 linear straight chain or branched alkyl such as butyl such as tertiary-butyl.

22. The compound of claim 1, wherein one or both of R4 and R5 are an optionally substituted C3-C15 cycloalkyl such as adamantyl.

23. The compound of claim 1, wherein R7 is hydrogen or optionally substituted C1-C8 linear straight chain or branched alkyl such as (CH2)_{y}OR′ and (CH2)_{y}NR′2, where each R′ is independently hydrogen, optionally substituted C1-C8 linear straight chain or branched alkyl or optionally substituted phenyl and y is an integer from 1 to 4, for example 1 or 2.

24. The compound of claim 1, wherein R7 is methyl, ethyl or propyl (e.g. isopropyl).

25. A method of catalyzing a reaction, comprising conducting the reaction in presence of a compound having the formula:
L-M-Xₙ
as a catalyst in a chemical reaction, wherein:
wherein:
M is a metal atom:
L is a ligand according to formula (I) 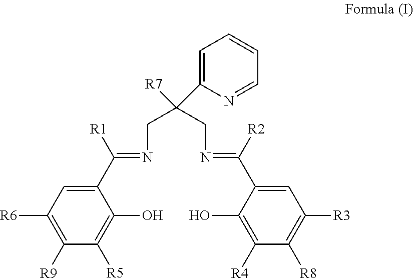
wherein,
R1 and R2 are the same or different and are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8)-alkyl where V is selected from a single bond, O, S and NH, phenyl-(C1-C8)-alkyl-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)PC—, (OR′)3C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC-(thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S, R′₃P (phosphino), (OH)₂(O)P-(phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3, R4, R5, R6, R8 and R9 are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8)-alkyl where V is selected from a single bond, O, S and NH, phenyl-(C1-C8)-alkyl-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)2C—, (OR′)3C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R₄N⁺— (quaternary amine), R′₂N—C(O)-(amide), R′(NR′)—C— (imine), R′(CO)NR′—C(O)— (imide), N, (azide), R′N₂— (azo), —SH (thiol), (OH)(O)S— (sulfino), (O)(O)(OH)S— (sulfo), thiocyanate (—SCN or —NCS), (S)HC-(thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)2(O)P-(phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R7 is hydrogen, optionally substituted C1-C8 linear straight chain or branched alkyl such as (CH₂)_{y}OR′ and (CH₂)_{y}NR′₂, CN (nitrile) and R′₃N— (amine), where each R′ is independently hydrogen, hydroxyl, halogen, optionally substituted C1-C8 alkoxyl, optionally substituted C1-C8 linear straight chain or branched alkyl, optionally substituted phenyl, optionally substituted C2-C8 alkenyl or optionally substituted C2-C8 alkynyl, and y is an integer from 1 to 8;
X is a co-ligand to balance the charge of the compound; and
n is an integer from 0 to 7.

26. The method of claim 25, wherein the chemical reaction is a ring-opening polymerization reaction or an isocyanate trimerization.

27. A method of making a compound having the formula: L-M-Xₙ, comprising base condensation of a pyridyl-diamine compound with an aldehyde or ketone,
wherein:
M is a metal atom;
L is a ligand according to formula (I) 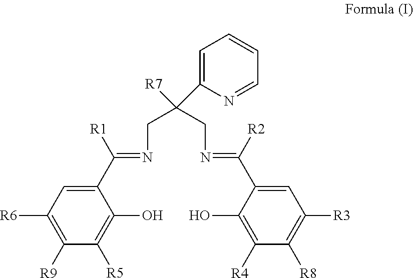
wherein,
R1 and R2 are the same or different and are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl, optionally substituted furyl, optionally substituted phenoxy, phenyl-V—(C1-C8)-alkyl where V is selected from a single bond, O, S and NH, phenyl-(C1-C8)-alkyl-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)3C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)— (amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC-(thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P— (phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R3, R4, R5, R6, R8 and R9 are each independently selected from hydrogen, halogen, optionally substituted C1-C8 alkoxy, optionally substituted C1-C8 linear straight chain or branched alkyl, optionally substituted C3-C15 cycloalkyl, optionally substituted C2-C8 alkenyl, optionally substituted C2-C8 alkynyl, optionally substituted phenyl which is optionally fused to the phenol ring in formula (I) to form a naphthalene moiety, optionally substituted furyl, optionally substituted phenoxy, phenyl-V—(C1-C8)-alkyl where V is selected from a single bond, O, S and NH, phenyl-(C1-C8)-alkyl-V— where V is selected from a single bond, O, S and NH, optionally substituted pyridyl, optionally substituted pyrimidinyl, optionally substituted pyrrolidinyl, optionally substituted piperidinyl, optionally substituted morpholino, optionally substituted piperazinyl, optionally substituted imidazoyl, R′—C(O)—, R′—C(O)—O—, R′—O—, R′₂N—C(O)—O—, R′(OR′)₂C—, (OR′)₃C—, (OR′)₃C—O—, nitro (—NO₂), nitrate (—ONO₂), nitrile (CN), nitrite (—ONO), nitroso (NO), cyano (—OCN or —NCO), optionally substituted pyridyl, R′(NOH)C— (oxime), R′₃N— (amine), R′₄N⁺— (quaternary amine), R′₂N—C(O)-(amide), R′(NR′)—C— (imine), R′(CO)NR′—C(O)— (imide), N₃ (azide), R′N₂— (azo), —SH (thiol), (OH)(O)S— (sulfino), (O)(O)(OH)S— (sulfo), thiocyanate (—SCN or —NCS), (S)HC-(thial), R′S— (sulfide), R′S—S— (disulphide), R′(O)S— (sulfoxide), (O)(O)R′S— (sulfonyl), R′SC (thioketone), R′S—C(O)— (thioester), R′C(O)—S—, R′₃P (phosphino), (OH)₂(O)P-(phosphono), (OH)₂(O)P—O— (phosphate) and (OR′)₂(O)P—O— (phosphoester), where each R′ is independently hydrogen, hydroxyl, halogen, optionally substituted C1-C8 alkoxyl, optionally substituted C1-C8 linear straight chain or branched alkyl, optionally substituted C2-C8 alkenyl or optionally substituted C2-C8 alkynyl;
R7 is hydrogen, optionally substituted C1-C8 linear straight chain or branched alkyl such as (CH2)_{y}OR′ and (CH2)_{y}NR′2, CN (nitrile) and R′sN— (amine), where each R′ is independently hydrogen, hydroxyl, halogen, optionally substituted C1-C8 alkoxyl, optionally substituted C1-C8 linear straight chain or branched alkyl, optionally substituted phenyl, optionally substituted C2-C8 alkenyl or optionally substituted C2-C8 alkynyl, and y is an integer from 1 to 8;
X is a co-ligand to balance the charge of the compound; and
n is an integer from 0 to 7.

28. (canceled)
